(19) United States
(12) Patent Application Publication
(10) Pub. No.: US 2025/0312368 A1
(43) Pub. Date: Oct. 9, 2025

(54) SAPONIN-BASED IMMUNOSTIMULANTS, PHARMACEUTICAL COMPOSITION COMPRISING SAID IMMUNOSTIMULANTS, THERAPEUTIC USE THEREOF

(71) Applicant: THE UAB RESEARCH FOUNDATION, BIRMINGHAM, AL (US)

(72) Inventor: Pengfei Wang, Hoover, AL (US)

(21) Appl. No.: 18/866,118

(22) PCT Filed: May 11, 2023
(86) PCT No.: PCT/US2023/066884

§ 371 Date: Nov. 15, 2024

Related U.S. Application Data
(60) Provisional application No. 63/364,897, filed on May 18, 2022.

(51) Int. Cl. A61K 31/704; A61K 39/00; A61K 39/39; A61K 45/06

(52) U.S. Cl. CPC A61K 31/704; A61K 39/39; A61K 45/06; A61K 2039/55577

(57) ABSTRACT

The present disclosure provides for compounds including modified saponin compounds, pharmaceutical compositions including modified saponin compounds, methods of use of the modified saponin compounds and the pharmaceutical compositions, methods of making modified saponin compounds, and the like. Compounds and pharmaceutical compositions of the present disclosure can be used in combination with one or more other therapeutic agents for treating viral infection and other diseases.

### CLAIM OF PRIORITY TO RELATED APPLICATION

This application claims priority to co-pending U.S. provisional application entitled “SAPONIN-BASED IMMUNOSTIMULANTS, PHARMACEUTICAL COMPOSITION COMPRISING SAID IMMUNOSTIMULANTS, THERAPEUTIC USE THEREOF” having Ser. No. 63/364,897 filed on May 18, 2022, which is entirely incorporated herein by reference.

### STATEMENT ON FUNDING PROVIDED BY THE U.S. GOVERNMENT

This invention was made with Government support under contract R01 GM120159 awarded by the National Institutes of Health. The Government has certain rights in the invention.

### BACKGROUND

Vaccine adjuvants are the substances used with a vaccine to potentiate host's immune responses to the specific antigen(s) introduced by the vaccine (Brunner et al., (2010) Immunol. Lett. 128:29-35; Kensil et al., (2004) Frontiers Biosci. 9:2972-2988; Leroux-Roels G. (2010) Vaccine 28 (Suppl 3): C25-36; Sharp & Lavelle (2012) Development Therapeutic Agents Handbook John Wiley & Sons, Inc; pp. 533-546; Wang W. (2011) World J. Vaccines 1:33-78; Weeratna & McCluskie (2011) Recent Advan. Vaccine Adjuvants. pp. 303-322; Cox & Coulter (1997) Vaccine 15:248-256; Klebanoff et al., (2010) Immunol. Rev. 239: 27-44; Plotkin S A. (2005) Nat. Med. 11: S5-S11; Rappuoli & Aderem (2011) Nature 473:463-469; Kensil et al., (2005) Vaccine Adjuvants: Immunological and Clinical Principles Humana Press Inc. pp. 221-234).

Vaccine adjuvants also tune immune system to the desirable responses for certain pathogens. For example, QS-21, a mixture of two isomers, is an FDA-approved adjuvant known for its capacity of potentiating a balanced Th1/Th2 response with antigen-specific CTL production, which is valuable for vaccines against intracellular pathogens and cancers (Ragupathi et al., (2011) Expert Rev. Vaccines 10:463-470; Deng et al., (2008) Angew Chem. Int. 47:6395-6398; Kensil C R. (1996) Critical Revs. Therap. Drug Carrier Systs. 13:1-55; Kensil et al., (1991) J. Immun. 146:431-437). It has potential for a wide range of clinical applications and thus to be in high demand (Kensil et al., (1991) J. Immun. 146:431-437). Supplies of QS-21 are very limited. The natural products are isolated from the tree bark of Quillaja saponaria Molina (QS), an evergreen tree native to warm temperate central Chile. However, overexploitation of the natural source has resulted in ecological and economic consequences even under the current demand. Moreover, the abundance of QS-21 in QS tree bark extracts is low and its isolation is laborious (Kensil et al., (1991) J. Immun. 146:431-437; Ragupathi et al., (2010) Vaccine 28:4260-4267; Wang et al., (2005) J. Am. Chem. Soc. 127:3256-3257). QS-21 also has a chemical instability issue due to two hydrolytically unstable ester moieties that complicate its formulation; its dose-limiting toxicity also prevents it from reaching the full potency.

Derivatization of Momordica saponin (MS) I and II has been shown to be a potentially viable way to access practical alternatives to QS-21 (Wang et al., (2019) J. Med. Chem. 62: 9976-9982). MS I and II are isolated from the seeds of the perennial Momordica cochinchinensis Spreng (MC) that grows in China and Southeast Asia. Derivatization of MS I/II at the C3 glucuronic acid can lead to immunostimulants with similar adjuvanticity to that of QS-21.

### SUMMARY

The present disclosure provides for compounds including modified saponin compounds, pharmaceutical compositions including modified saponin compounds, methods of use of the modified saponin compounds and the pharmaceutical compositions, methods of making modified saponin compounds, and the like.

The present disclosure provides for a modified saponin having the formula:

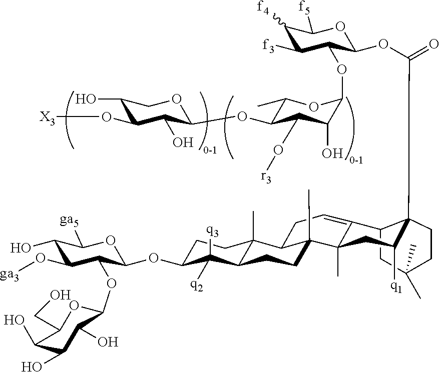

wherein:
q₁ can be H or OH;
q₂ and q₃ are each independently selected from CHO, CH₃, CH₂OH, H, COOH, a component of an acetal group, or an imine group;
f₃ and f₄ are each independently OH or an acetyl, or C3 and C4 of a fuocsyl unit wherein f₃, and f₄ can form a cyclic ketal ring or cyclic carbonate ester;
f₅ is selected from H, methyl group, —CH₂OH group, R₄—NR₅—C(O)—, R₄—O—C(O)—, or R₄—O—CH₂—;
wherein R₄ and R₅ are each independently a linear chain having the structure R₆[(CX₂)₀₋₂₀O₀₋₁(CY₂)₀₋₂₀]₀₋₂₀;
wherein X and Y are each independently H or a halogen atom;
wherein R₆ are COOR₇, C(O)NR₇—R₈, NR₇R₈, or OR₇;
wherein R₇ are Ar[(CZ₂)₀₋₂₀O₀₋₁(CL₂)₀₋₂₀]₀₋₂₀, wherein R₈ is H or an alkyl;
wherein Z and L are each independently H or a halogen atom;
wherein Ar is a substituted or unsubstituted aromatic system;
ga₅ is selected from R₁₄—NR₁₅—C(O)—, R₁₄—O—C(O)—, or R₁₄—O—CH₂—;
wherein R₁₄ and R₁₅ are each independently a linear chain having the structure R₁₆[(CX1₂)₀₋₂₀O₀₋₁(CY1₂)₀₋₂₀]⁰⁻²⁰;
wherein X1 and Y1 are each independently H or a halogen atom;
wherein R₁₆ is COOR₁₇, C(O)NR₁₇R₁₈, NR₁₇R₁₈, or OR₁₇;
wherein R₁₇ is Ar2[(CZ1₂)₀₋₂₀O₀₋₁(CL1₂)₀₋₂₀]₀₋₂₀, wherein R₁₈ is H or an alkyl;
wherein Z1 and L1 are each independently H or a halogen atom;
wherein Ar2 is a substituted or unsubstituted aromatic group;
r3 is H, a monosaccharide, disaccharide, or a trisaccharide;
x3 is H, a monosaccharide (except xylose) or a disaccharide; and
ga3 is H, a monosaccharide or a disaccharide.

The present disclosure provides for a modified saponin can have the formula I:

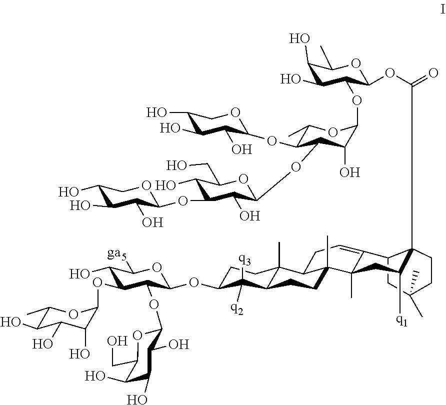

wherein:
q₁ is H or OH;
q₂ and q₃ are each independently selected from CHO, CH₃, CH₂OH, H, COOH, a component of an acetal group, or an imine group;
ga₅ is selected from R₁₄—NR₁₅—C(O)—, R₁₄—O—C(O)—, or R₁₄—O—CH₂—;
wherein R₁₄ and R₁₅ are each independently a linear chain having the structure R₁₆[(CX1₂)₀₋₂₀O₀₋₁(CY1₂)₀₋₂₀]₀₋₂₀;
wherein X1 and Y1 are each independently H or a halogen atom;
wherein R₁₆ is COOR₁₇, C(O)NR₁₇R₁₈, NR₁₇R₁₈, or OR₁₇;
wherein R₁₇ is Ar2[CZ1₂)₀₋₂₀O₀₋₁(CL1₂)₀₋₂₀]₀₋₂₀, wherein R₁₈ is H or an alkyl;
wherein Z1 and L1 are each independently H or a halogen atom; and
wherein Ar2 is a substituted or unsubstituted aromatic group.

The present disclosure provides for a modified saponin that can be selected from the group consisting of formulas 2D-2H:

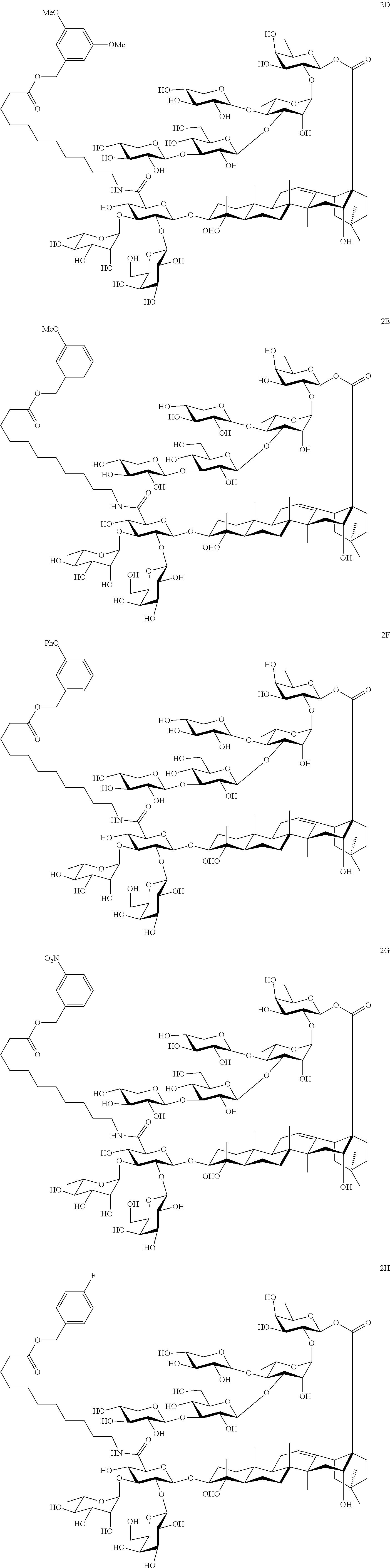

The present disclosure provides for a pharmaceutical composition comprising a therapeutically effective amount of a modified saponin having the formula, to treat a condition:

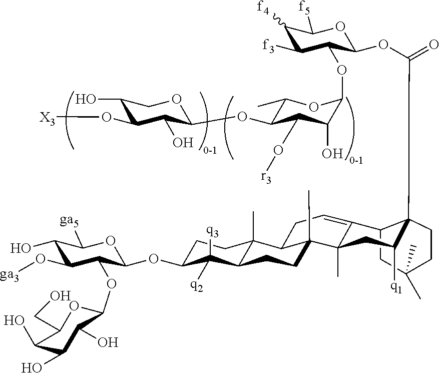

wherein:
q₁ is H or OH;
q₂ and q₃ are each independently selected from CHO, CH₃, CH₂OH, H, COOH, a component of an acetal group, or an imine group;
f₃ and f₄ are each independently OH or an acetyl, or C3 and C4 of a fuocsyl unit wherein f₃, and f₄ can form a cyclic ketal ring or cyclic carbonate ester;
f₅ is selected from H, methyl group, —CH₂OH group, R₄—NR₅—C(O)—, R₄—O—C(O)—, or R₄—O—CH₂—;
wherein R₄ and R₅ are each independently a linear chain having the structure R₆[(CX₂)₀₋₂₀O₀₋₁(CY₂)₀₋₂₀]₀₋₂₀;
wherein X and Y are each independently H or a halogen atom;
wherein R₆ are COOR₇, C(O)NR₇R₈, NR₇R₈, or OR₇;
wherein R₇ are Ar[(CZ₂)₀₋₂₀O₀₋₁(CL₂)₀₋₂₀]₀₋₂₀, wherein R₈ is H or an alkyl;
wherein Z and L are each independently H or a halogen atom;
wherein Ar is a substituted or unsubstituted aromatic system;
ga₅ is selected from R₁₄—NR₁₅—C(O)—, R₁₄—O—C(O)—, or R₁₄—O—CH₂—;
wherein R₁₄ and R₁₅ are each independently a linear chain having the structure R₁₆[(CX1₂)₀₋₂₀O₀₋₁(CY1₂)₀₋₂₀]₀₋₂₀;
wherein X1 and Y1 are each independently H or a halogen atom;
wherein R₁₆ is COOR₁₇, C(O)NR₁₇R₁₈, NR₁₇R₁₈, or OR₁₇;
wherein R₁₇ is Ar2[(CZ1₂)₀₋₂₀O₀₋₁(CL1₂)₀₋₂₀]₀₋₂₀, wherein R₁₈ is H or an alkyl;
wherein Z1 and L1 are each independently H or a halogen atom;
wherein Ar2 is a substituted or unsubstituted aromatic group;
r3 is H, a monosaccharide, disaccharide, or a trisaccharide;
x3 is H, a monosaccharide (except xylose) or a disaccharide; and
ga3 is H, a monosaccharide or a disaccharide.

The present disclosure provides for a pharmaceutical composition comprising a therapeutically effective amount of a modified saponin having the formula I, to treat a condition:

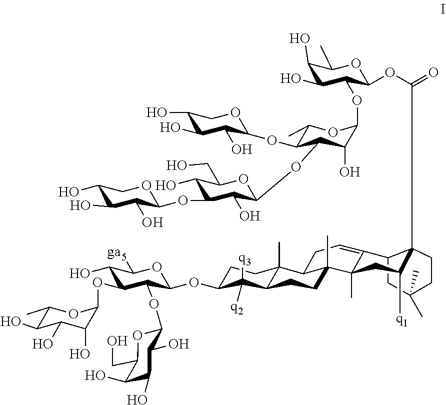

wherein:
q₁ is H or OH;
q₂ and q₃ are each independently selected from CHO, CH₃, CH₂OH, H, COOH, a component of an acetal group, or an imine group;
ga₅ is selected from R₁₄—NR₁₅—C(O)—, R₁₄—O—C(O)—, or R₁₄—O—CH₂—;
wherein R₁₄ and R₁₅ are each independently a linear chain having the structure R₁₆[(CX1₂)₀₋₂₀O₀₋₁(CY1₂)₀₋₂₀]₀₋₂₀;
wherein X1 and Y1 are each independently H or a halogen atom;
wherein R₁₆ is COOR₁₇, C(O)NR₁₇R₁₈, NR₁₇R₁₈, or OR₁₇;
wherein R₁₇ is Ar2[(CZ1₂)₀₋₂₀O₀₋₁(CL1₂)₀₋₂₀]₀₋₂₀, wherein R₁₈ is H or an alkyl;
wherein Z1 and L1 are each independently H or a halogen atom; and
wherein Ar2 is a substituted or unsubstituted aromatic group.

The present disclosure provides for a therapeutically effective amount of a modified saponin having one of formulas 2D-H, to treat a condition:

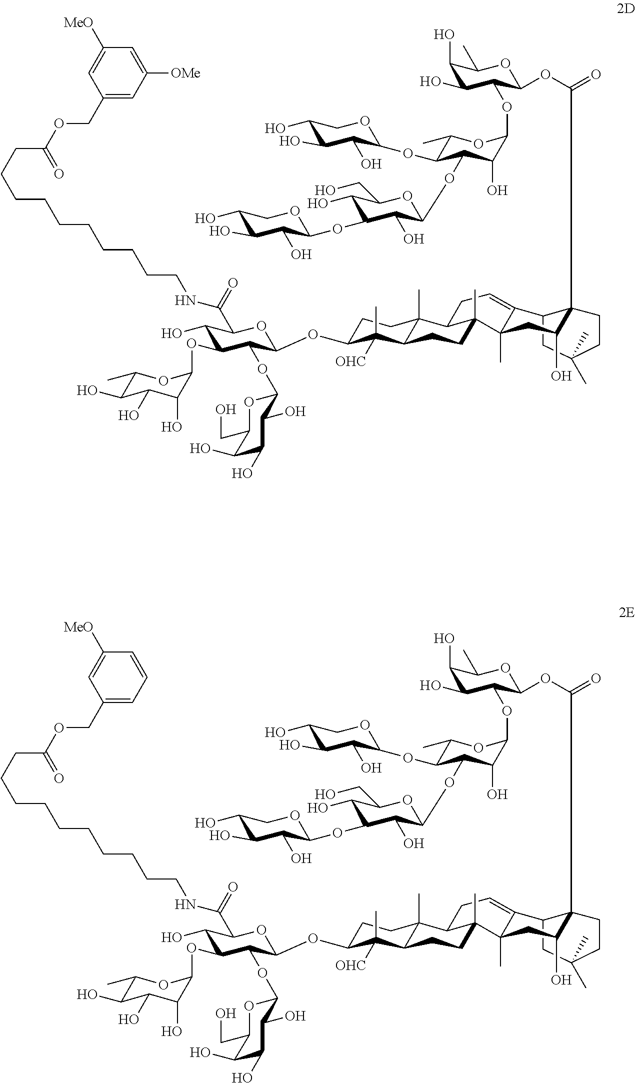

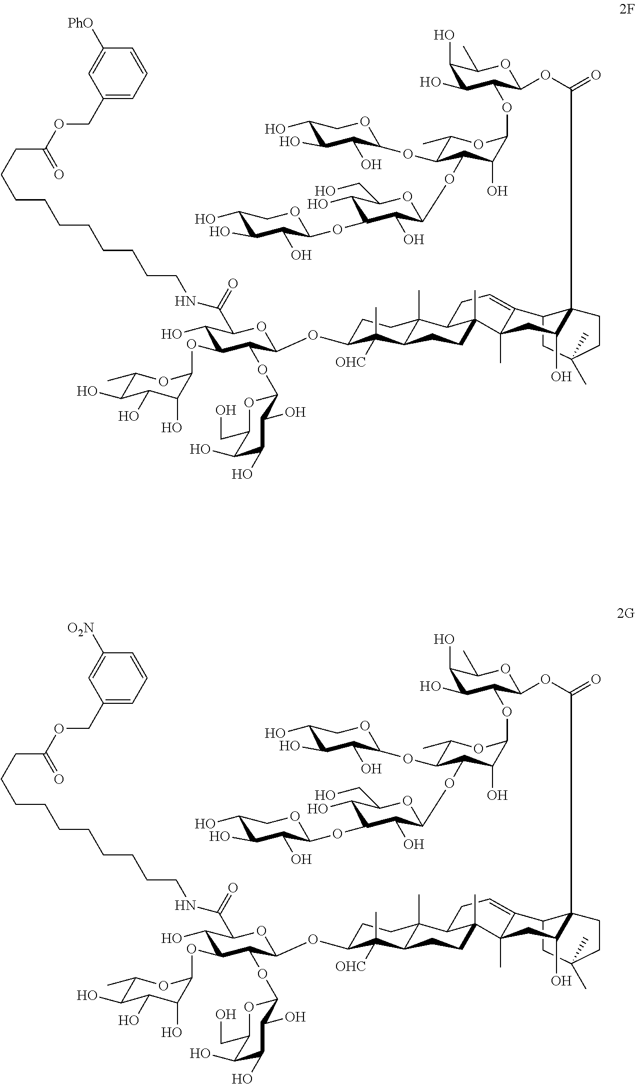

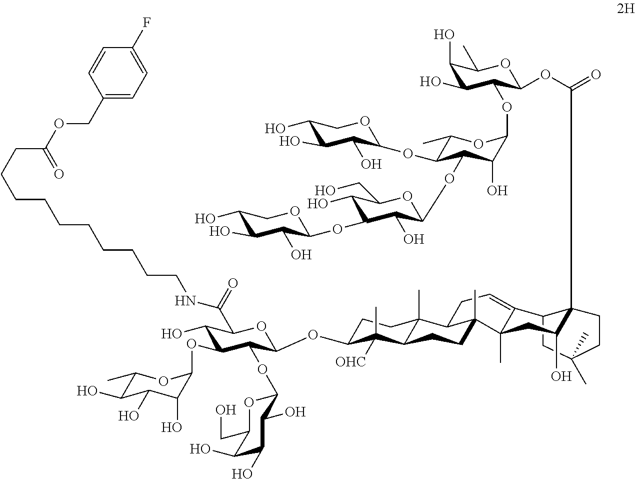

The present disclosure provides for pharmaceutical composition, such as those described above, that can also include at least one immunogen, a pharmaceutically acceptable carrier, at least one therapeutic agent (e.g., at least one cancer therapeutic agent). The pharmaceutical composition can be formulated for administration to an animal or human subject.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 illustrates preparation of MS derivatives 1D-1H from MS II.

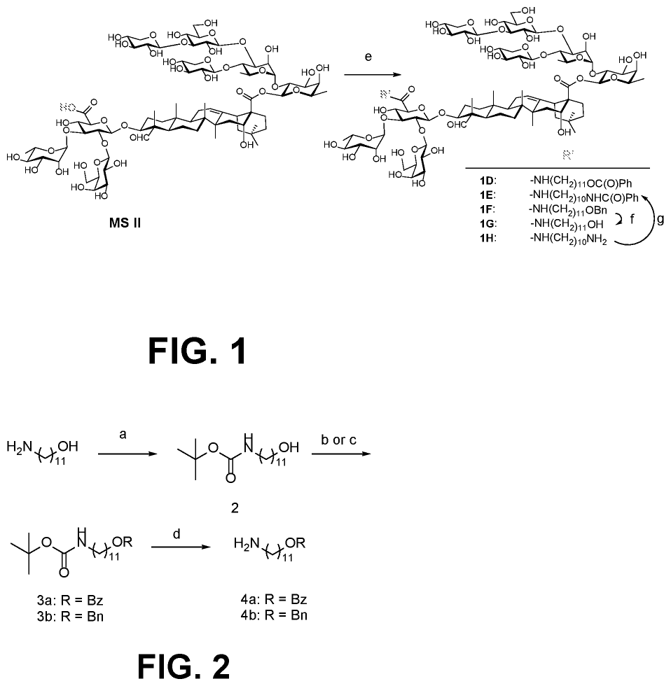

FIG. 2 illustrates the synthesis of the side chains to be incorporated into 1D-1H.

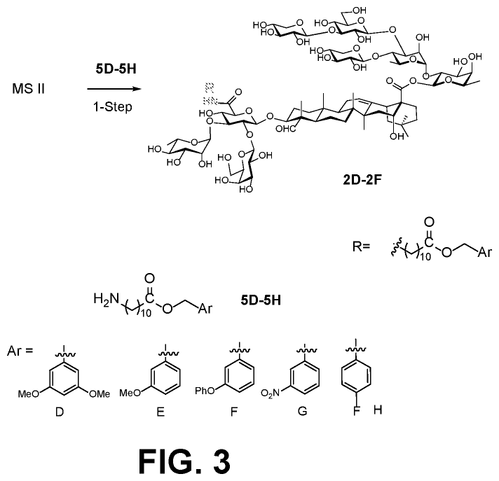

FIG. 3 illustrates preparation of MS derivatives 2D-2H from MS II.

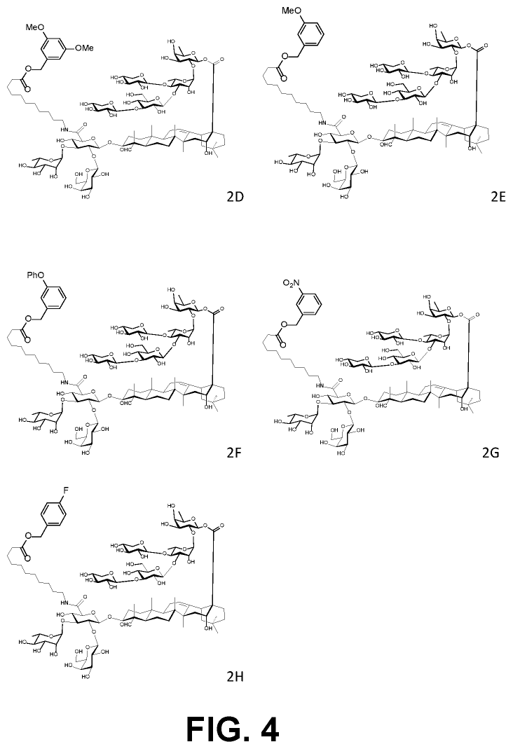

FIG. 4 illustrates derivatives 2D-2H of the disclosure.

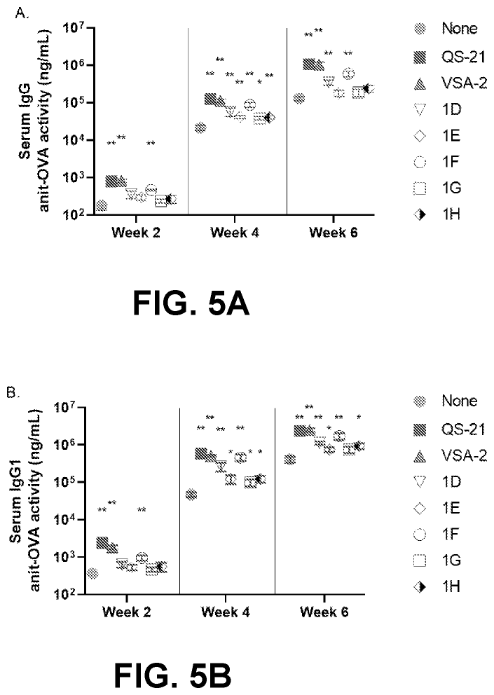

FIGS. 5A-5C illustrate serum IgG, IgG1, and IgG2a anti-OVA responses in mice immunized by the subcutaneous route with ovalbumin (OVA) alone, or with QS-21 or VSA-2, or with one of the saponin adjuvants 1D-1H. Statistical significance was evaluated by t tests (with unpaired, nonparametric and Mann-Whiteny test). *P<0.05, **P<0.01.

FIGS. 6A-6C illustrate serum IgG, IgG1, and IgG2a anti-OVA responses in mice immunized by the subcutaneous route with ovalbumin (OVA) alone, or with QS-21 or VSA-2, or with one of the saponin adjuvants 2D-2H.

### DETAILED DESCRIPTION

This disclosure is not limited to particular embodiments described, and as such may, of course, vary. The terminology used herein serves the purpose of describing particular embodiments only, and is not intended to be limiting, since the scope of the present disclosure will be limited only by the appended claims.

Where a range of values is provided, each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, dimensions, frequency ranges, applications, or the like, as such can vary. It is also to be understood that the terminology used herein is for purposes of describing particular embodiments only, and is not intended to be limiting. It is also possible in the present disclosure that steps can be executed in different sequence, where this is logically possible. It is also possible that the embodiments of the present disclosure can be applied to additional embodiments involving measurements beyond the examples described herein, which are not intended to be limiting. It is furthermore possible that the embodiments of the present disclosure can be combined or integrated with other measurement techniques beyond the examples described herein, which are not intended to be limiting.

It should be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. Further, documents or references cited in this text, in a Reference List before the claims, or in the text itself; and each of these documents or references (“herein cited references”), as well as each document or reference cited in each of the herein-cited references (including any manufacturer's specifications, instructions, etc.) are hereby expressly incorporated herein by reference.

Prior to describing the various embodiments, the following definitions are provided and should be used unless otherwise indicated.

### Definitions

It is understood that “substitution” or “substituted” includes the implicit proviso that such substitution is in accordance with permitted valence of the substituted atom and the substituent, and that the substitution results in a stable compound, i.e., a compound that does not spontaneously undergo transformation such as by rearrangement, cyclization, elimination, etc.

It will be understood by those skilled in the art that the moieties substituted can themselves be substituted, if appropriate. For instance, the substituents of a substituted alkyl may include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Cycloalkyls can be substituted in the same manner.

The term “acyl” as used herein, alone or in combination, means a carbonyl or thiocarbonyl group bonded to a radical selected from, for example, optionally substituted, hydrido, alkyl (e.g. haloalkyl), alkenyl, alkynyl, alkoxy (“acyloxy” including acetyloxy, butyryloxy, iso-valeryloxy, phenylacetyloxy, berizoyloxy, p-methoxybenzoyloxy, and substituted acyloxy such as alkoxyalkyl and haloalkoxy), aryl, halo, heterocyclyl, heteroaryl, sulfonyl (e.g. allylsulfinylalkyl), sulfonyl (e.g. alkylsulfonylalkyl), cycloalkyl, cycloalkenyl, thioalkyl, thioaryl, amino (e.g alkylamino or dialkylamino), and aralkoxy. Illustrative examples of “acyl” radicals are formyl, acetyl, 2-chloroacetyl, 2-bromacetyl, benzoyl, trifluoroacetyl, phthaloyl, malonyl, nicotinyl, and the like. The term “acyl” as used herein refers to a group —C(O)R₂₆, where R₂₆ is hydrogen, alkyl, cycloalkyl, cycloheteroalkyl, aryl, arylalkyl, heteroalkyl, heteroaryl, and heteroarylalkyl. Examples include, but are not limited to formyl, acetyl, cyclohexylcarbonyl, cyclohexylmethylcarbonyl, benzoyl, beozylcarbonyl and the like.

The term “adjuvant molecule” as used herein refers to surface proteins capable of eliciting an immune response in a host. In particular embodiments, the adjuvant molecule is a “membrane-anchored form” of the adjuvant molecule which indicates that the adjuvant molecule has been engineered to include a signal peptide (SP) and a membrane anchor sequence to direct the transport and membrane orientation of the protein. Thus, in embodiments, a membrane-anchored form of an adjuvant molecule is a recombinant protein including a portion of a protein fused to a SP and membrane anchor sequence.

The terms “administering” and “administration” as used herein refer to introducing a composition (e.g., a vaccine, adjuvant, or immunogenic composition) of the present disclosure into a subject. As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. A preferred route of administration of the vaccine composition is intravenous.

The terms “alkoxyl” or “alkoxyalkyl” as used herein refer to an alkyl-O— group wherein alkyl is as previously described. The term “alkoxyl” as used herein can refer to C₁₋₂₀ inclusive, linear, branched, or cyclic, saturated or unsaturated oxo-hydrocarbon chains, including, for example, methoxyl, ethoxyl, propoxyl, isopropoxyl, butoxyl, t-butoxyl, and pentoxyl.

The term “alkyl”, either alone or within other terms such as “thioalkyl” and “arylalkyl”, as used herein, means a monovalent, saturated hydrocarbon radical which may be a straight chain (i.e. linear) or a branched chain. An alkyl radical for use in the present disclosure generally comprises from about 1 to 20 carbon atoms, particularly from about 1 to 10, 1 to 8 or 1 to 7, more particularly about 1 to 6 carbon atoms, or 3 to 6. Illustrative alkyl radicals include methyl, ethyl, n-propyl, n-butyl, n-pentyl, n-hexyl, isopropyl, isobutyl, isopentyl, amyl, sec-butyl, tert-butyl, tert-pentyl, n-heptyl, n-actyl, n-nonyl, n-decyl, undecyl, n-dodecyl, n-tetradecyl, pentadecyl, n-hexadecyl, heptadecyl, n-octadecyl, nonadecyl, eicosyl, dosyl, n-tetracosyl, and the like, along with branched variations thereof. In certain aspects of the disclosure an alkyl radical is a C₁-C₆ lower alkyl comprising or selected from the group consisting of methyl, ethyl, n-propyl, n-butyl, n-pentyl, n-hexyl, isopropyl, isobutyl, isopentyl, amyl, tributyl, sec-butyl, tert-butyl, tert-pentyl, and n-hexyl. An alkyl radical may be optionally substituted with substituents as defined herein at positions that do not significantly interfere with the preparation of compounds of the disclosure and do not significantly reduce the efficacy of the compounds. In certain aspects of the disclosure, an alkyl radical is substituted with one to five substituents including halo, lower alkoxy, lower aliphatic, a substituted lower aliphatic, hydroxy, cyano, nitro, thio, amino, keto, aldehyde, ester, amide, substituted amino, carboxyl, sulfonyl, sulfuryl, sulfenyl, sulfate, sulfoxide, substituted carboxyl, halogenated lower alkyl (e.g. CF₃), halogenated lower alkoxy, hydroxycarbonyl, lower alkoxycarbonyl, lower alkylcarbonyloxy, lower alkylcarbonylamino, cycloaliphatic, substituted cycloaliphatic, or aryl (e.g., phenylmethyl benzyl)), heteroaryl (e.g., pyridyl), and heterocyclic (e.g., piperidinyl, morpholinyl). Substituents on an alkyl group may themselves be substituted.

The Ar (e.g., Ar₁, Ar₂, etc) group is an aromatic system or group such as an aryl group. “Aryl”, as used herein, refers to C₅-C₂₀-membered aromatic, heterocyclic, fused aromatic, fused heterocyclic, biaromatic, or bihetereocyclic ring systems. In an aspect, “aryl”, can include 5-, 6-, 7-, 8-, 9-, and 10-membered single-ring aromatic groups that may include from zero to four heteroatoms, for example, functional groups that correspond to benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with one or more substituents including, but not limited to, halogen, azide, alkyl, aralkyl, alkenyl, alkynyl, cycloalkyl, hydroxyl, alkoxyl, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN; and combinations thereof.

The term “aryl” also includes polycyclic ring systems (C₅-C₃₀) having two or more cyclic rings in which two or more carbons are common to two adjoining rings (i.e., “fused rings”) wherein at least one of the rings is aromatic, e.g., the other cyclic ring or rings can be cycloalkyls, cycloalkenyls, cycloalkynyls, aryls and/or heterocycles. Examples of heterocyclic rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined above for “aryl”.

The term “antibody” as used herein refers to polyclonal and monoclonal antibody preparations, as well as preparations including hybrid antibodies, altered antibodies, F(ab′)₂ fragments, F(ab) fragments, Fv fragments, single domain antibodies, chimeric antibodies, humanized antibodies, and functional fragments thereof which exhibit immunological binding properties of the parent antibody molecule.

The term “antibody” as used herein further refers to an immunoglobulin which specifically binds to and is thereby defined as complementary with a particular spatial and polar organization of another molecule. The antibody can be monoclonal, polyclonal, or a recombinant antibody, and can be prepared by techniques that are well known in the art such as immunization of a host and collection of sera (polyclonal) or by preparing continuous hybrid cell lines and collecting the secreted protein (monoclonal), or by cloning and expressing nucleotide sequences, or mutagenized versions thereof, coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b and IgG3, IgM, IgY, etc. Fragments thereof may include Fab, Fv and F(ab′)₂, Fab′, scFv, and the like. In addition, aggregates, polymers, and conjugates of immunoglobulins or their fragments can be used where appropriate so long as binding affinity for a particular molecule is maintained.

The term “antigen” as used herein refers to a molecule with one or more epitopes that stimulate a host's immune system to make a secretory, humoral and/or cellular antigen-specific response, or to a DNA molecule that is capable of producing such an antigen in a vertebrate. The term is also used interchangeably with “immunogen.” For example, a specific antigen can be complete protein, portions of a protein, peptides, fusion proteins, glycosylated proteins and combinations thereof. For use with the compositions of the present disclosure, one or more PvDBPII antigens (native protein or protein fragment), may be provided directly or as part of a recombinant nucleic acid expression system to provide an antigenic PvDBPII product to trigger a host immune response.

The term “antigenic component” as used herein refers to a component derived from an organism capable of stimulating an immune response in an animal, preferably a mammal including mouse and human. An antigenic component may be an immunogenic agent. The antigenic component may comprise sub-cellular components including, organelles, membranes, proteins, lipids, glycoproteins and other components derived from the organism. The antigenic component may be derived from a whole organism, for example a whole parasite, or a part of an organism, for example a cell or tissue of an organism. Also, a sub-set of proteins may be purified, for example by size fractionation or affinity purification, and recombined.

The terms “sugar” and “saccharide” as used herein refers to a polyhydroxyaldehyde, polyhydroxyketone and derivatives thereof. The term includes monosaccharides such as erythrose, arabinose, allose, altrose, glucose, mannose, threose, xylose, gulose, idose, galactose, talose, aldohexose, fructose, ketohexose, ribose, and aldopentose. The term also includes carbohydrates composed of monosaccharide units, including disaccharides, oligosaccharides, or polysaccharides. Examples of disaccharides are sucrose, lactose, and maltose. Oligosaccharides generally contain between 3 and 9 monosaccharide its and polysaccharides contain greater than 10 monosaccharide units. A sugar may be a member of the D or L series and can include amino sugars, deoxy sugars, and their uronic acid derivatives. In embodiments of the disclosure where the carbohydrate is a hexose, the hexose is glucose, galactose, or mannose, or substituted hexose sugar residues such as an amino sugar residue such as hexosamine, galactosamine; glucosamine, in particular D-glucosamine (2-amino-2-doexy-D-gluoose) or D-galactosamine (2-amino-2-deoxy-D-galactose). Illustrative pentose sugars include arabinose, fucose, and ribose. A sugar residue may be linked to a compound of the disclosure from a 1,1 linkage, 1,2 linkage, 1,3 linkage, 1,4 linkage, 1,5 linkage, or 1,6 linkage. A linkage may be via an oxygen atom of a compound of the disclosure. An oxygen atom can be replaced one or more times by —CH₂— or —S— groups.

The term “carboxyl” as used herein, alone or in combination, refers to —C(O)OR₂₅— or —C(—O)OR₂₅ wherein R₂₅ is hydrogen, alkyl, alkenyl, alkynyl, cycloalkyl, cycloalkenyl, amino, thiol, aryl, heteroaryl, thioalkyl, thioaryl, thioalkoxy, a heteroaryl, or a heterocyclic, which may optionally be substituted. In aspects of the disclosure, the carboxyl groups are in an esterified form and may contain as an esterifying group lower alkyl groups. In particular aspects of the disclosure, —C(O)OR₂₅ provides an ester or an amino acid derivative. An esterified form is also particularly referred to herein as a “carboxylic ester”. In aspects of the disclosure a “carboxyl” may be substituted, in particular substituted with allyl which is optionally substituted with one or more of amino, amine, halo, alkylamino, aryl, carboxyl, or a heterocyclic. Examples of carboxyl groups are methoxycarbonyl, butoxycarbonyl, tert.alkoxycarbonyl such as tert-butoxycarbonyl, arylmethyoxycarbonyl having one or two aryl radicals including without limitation phenyl optionally substituted by for example lower alkyl, lower alkoxy, hydroxyl, halo, and/or nitro, such as benzyloxycarbonyl, methoxybenzyloxycarbonyl, diphenylmethoxycarbonyl, 2-bromoethoxycarbonyl, 2-iodoethoxycarbonyltert.butylcarborlyl, 4-nitrobenzyloxycarbonyl, diphenylmethoxy-carbonyl, benzhydroxycarbonyl, di-(4-methoxyphenyl-methoxycarbonyl, 2-bromoethoxycarbonyl, 2-iodoethoxycarbonyl, 2-trimethylsilylethoxycarbonyl, or 2-triphenylsilylethoxycarbonyl. Additional carboxyl groups in esterified form are silyloxycarbonyl groups including organic silyloxycarbonyl. The silicon substituent in such compounds may be substituted with lower alkyl (e.g. methyl), alkoxy (e.g. methoxy), and/or halo (e.g. chlorine). Examples of silicon substituents include trimethylsilyi and dimethyltert.butylsilyl. In aspects of the disclosure, the carboxyl group may be an alkoxy carbonyl, in particular methoxy carbonyl, ethoxy carbonyl, isopropoxy carbonyl, t-butoxycarbonyl, t-pentyloxycarbonyl, sir heptyloxy carbonyl, especially methoxy carbonyl or ethoxy carbonyl.

The term “imine” refers to R′₁—N═CR″R′″, where R′, R″, and R′″ are each independently selected from an alkyl, an alkenyl, a carbocycle group, a heterocyclo, an aryl, or a heteroaryl.

The term “composition” as used herein refers to a product comprising the specified ingredients in the specified amounts, as well as any product which results, directly or indirectly, from combination of the specified ingredients in the specified amounts. Such a term in relation to a pharmaceutical composition is intended to encompass a product comprising the active ingredient(s), and the inert ingredient(s) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure and a pharmaceutically acceptable carrier.

When a compound of the present disclosure is used contemporaneously with one or more other drugs, a pharmaceutical composition containing such other drugs in addition to the compound of the present disclosure is contemplated. Accordingly, the pharmaceutical compositions of the present disclosure include those that also contain one or more other active ingredients, in addition to a compound of the present disclosure. The weight ratio of the compound of the present disclosure to the second active ingredient may be varied and will depend upon the effective dose of each ingredient. Generally, an effective dose of each will be used. Thus, for example, but not intended to be limiting, when a compound of the present disclosure is combined with another agent, the weight ratio of the compound of the present disclosure to the other agent will generally range from about 1000:1 to about 1:1000, preferably about 200:1 to about 1:200. Combinations of a compound of the present disclosure and other active ingredients will generally also be within the aforementioned range, but in each case, an effective dose of each active ingredient should be used. In such combinations the compound of the present disclosure and other active agents may be administered separately or in conjunction. In addition, the administration of one element may be prior to, concurrent to, or subsequent to the administration of other agent(s).

A composition of the disclosure can be a liquid solution, suspension, emulsion, tablet, pill, capsule, sustained release formulation, or powder. The compositions can be formulated as a suppository, with traditional binders and carriers such as triglycerides. Oral formulations can include standard carriers such as pharmaceutical grades of mannitol, lactose, starch, magnesium stearate, sodium saccharine, cellulose, magnesium carbonate, etc. Various delivery systems are known and can be used to administer a composition of the disclosure, e.g. encapsulation in liposomes, microparticles, microcapsules, and the like.

A therapeutic composition of the disclosure may comprise a carrier, such as one or more of a polymer, carbohydrate, peptide or derivative thereof, which may be directly or indirectly covalently attached to the compound. A carrier may be substituted with substituents described herein including without limitation one or more alkyl, amino, nitro, halogen, thiol, thioalkyl, sulfate, sulfonyl, sulfinyl, sulfoxide, hydroxyl groups. In aspects of the disclosure the carrier is an amino acid including alanine, glycine, praline, methionine, serine, threonine, asparagine, alanyl-alanyl, prolyl-methionyl, or glycyl-glycyl. A carrier can also include a molecule that targets a compound of the disclosure to a particular tissue or organ.

Compounds of the disclosure can be prepared using reactions and methods generally known to the person of ordinary skill in the art, having regard to that knowledge and the disclosure of this application including the Examples. The reactions are performed in solvent appropriate to the reagents and materials used and suitable for the reactions being effected. It will be understood by those skilled in the art of organic synthesis that the functionality present on the compounds should be consistent with the proposed reaction steps. This will sometimes require modification of the order of the synthetic steps or selection of one particular process scheme over another in order to obtain a desired compound of the disclosure. It will also be recognized that another major consideration in the development of a synthetic route is the selection of the protecting group used for protection of the reactive functional groups present in the compounds described in this disclosure. An authoritative account describing the many alternatives to the skilled artisan is Greene and Wuts (Protective Groups In Organic Synthesis, Wiley and Sons, 1991).

A compound of the disclosure of the disclosure may be formulated into a pharmaceutical composition for administration to a subject by appropriate methods known in the art. Pharmaceutical compositions of the present disclosure or fractions thereof comprise suitable pharmaceutically acceptable carriers, excipients, and vehicles selected based on the intended form of administration, and consistent with conventional pharmaceutical practices. Suitable pharmaceutical carriers, excipients, and vehicles are described in the standard text, Remington: The Science and Practice of Pharmacy (21.sup.st Edition. 2005, University of the Sciences in Philadelphia (Editor), Mack Publishing Company), and in The United States Pharmacopeia: The National Formulary (USP 24 NF19) published in 1999. By way of example for oral administration in the form of a capsule or tablet, the active components can be combined with an oral, non-toxic pharmaceutically acceptable inert carrier such as lactose, starch, sucrose, methyl cellulose, magnesium stearate, glucose, calcium sulfate, dicalcium phosphate, mannitol, sorbital, and the like. For oral administration in a liquid form, the chug components may be combined with any oral, non-toxic, pharmaceutically, acceptable inert carrier such as ethanol, glycerol, water, and the like. Suitable binders (e.g., gelatin, starch, corn sweeteners, natural sugars including glucose; natural and synthetic gums, and waxes), lubricants (e.g. sodium oleate, sodium stearate, magnesium stearate, sodium benzoate, sodium acetate, and sodium chloride), disintegrating agents (e.g. starch, methyl cellulose, agar, bentonite, and xanthan gum), flavoring agents, and coloring agents may also be combined in the compositions or components thereof. Compositions as described herein can further comprise wetting or emulsifying agents, or pH buffering agents.

The term “immunogenic composition” as used herein are those which result in specific antibody production or in cellular immunity when injected into a host.

The immunogenic compositions and/or vaccines of the present disclosure may be formulated by any of the methods known in the art. They can be typically prepared as injectables or as formulations for intranasal administration, either as liquid solutions or suspensions. Solid forms suitable for solution in, or suspension in, liquid prior to injection or other administration may also be prepared. The preparation may also, for example, be emulsified, or the protein(s)/peptide(s) encapsulated in liposomes.

The active immunogenic ingredients are often mixed with excipients or carriers, which are pharmaceutically acceptable and compatible with the active ingredient. Suitable excipients include but are not limited to water, saline, dextrose, glycerol, ethanol, or the like and combinations thereof. The concentration of the immunogenic polypeptide in injectable, aerosol or nasal formulations is usually in the range of about 0.2 to 5 mg/ml. Similar dosages can be administered to other mucosal surfaces.

In addition, if desired, the vaccines may contain minor amounts of auxiliary substances such as wetting or emulsifying agents, pH buffering agents, and/or other agents, which enhance the effectiveness of the vaccine. Examples of agents which may be effective include, but are not limited to, aluminum hydroxide; N-acetyl-muramyl-L-threonyl-D-isoglutamine (thr-MDP); N-acetyl-nor-muramyl-L-alanyl-D-isoglutamine (CGP 11637, referred to as nor-MDP); N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE); and RIBI, which contains three components extracted from bacteria: monophosphoryl lipid A, trehalose dimycolate and cell wall skeleton (MPL+TDM+CWS) in a 2% squalene/Tween 80 emulsion. The effectiveness of the auxiliary substances may be determined by measuring the amount of antibodies (especially IgG, IgM or IgA) directed against the immunogen resulting from administration of the immunogen in vaccines which comprise the adjuvant in question. Additional formulations and modes of administration may also be used.

The immunogenic compositions and/or vaccines of the present disclosure can be administered in a manner compatible with the dosage formulation and in such amount and manner as will be prophylactically and/or therapeutically effective, according to what is known to the art. The quantity to be administered, which is generally in the range of about 1 to 1,000 micrograms of viral surface envelope glycoprotein per dose and/or adjuvant molecule per dose, more generally in the range of about 5 to 500 micrograms of glycoprotein per dose and/or adjuvant molecule per dose, depends on the nature of the antigen and/or adjuvant molecule, subject to be treated, the capacity of the host's immune system to synthesize antibodies, and the degree of protection desired. Precise amounts of the active ingredient required to be administered may depend on the judgment of the physician or veterinarian and may be peculiar to each individual, but such a determination is within the skill of such a practitioner.

The vaccine or immunogenic composition may be given in a single dose; two-dose schedule, for example, two to eight weeks apart; or a multi-dose schedule. A multi-dose schedule is one in which a primary course of vaccination may include 1 to 10 or more separate doses, followed by other doses administered at subsequent time intervals as required to maintain and/or reinforce the immune response (e.g., at 1 to 4 months for a second dose, and if needed, a subsequent dose(s) after several months).

The term “immunogenic fragment” as used herein refers to a fragment of an immunogen that includes one or more epitopes and thus can modulate an immune response or can act as an adjuvant for a co-administered antigen. Such fragments can be identified using any number of epitope mapping techniques, well known in the art (see, e.g., Epitope Mapping Protocols in Methods in Molecular Biology, Vol. 66 (Morris, G. E., Ed., 1996) Humana Press, Totowa, NJ).

Immunogenic fragments can be at least about 2 amino acids in length, more preferably about 5 amino acids in length, and most preferably at least about 10 to about 15 amino acids in length. There is no critical upper limit to the length of the fragment, which can comprise nearly the full-length of the protein sequence or even a fusion protein comprising two or more epitopes.

The term “immunoglobulin” as used herein refers to a class of proteins that exhibit antibody activity and bind to other molecules (e.g., antigens and certain cell-surface receptors) with a high degree of specificity. Immunoglobulins can be divided into five classes: IgM, IgG, IgA, IgD, and IgE. IgG is the most abundant antibody class in the body and assumes a twisted “Y” shape configuration. With the exception of the IgMs, immunoglobulins are composed of four peptide chains that are linked by intrachain and interchain disulfide bonds. IgGs are composed of two polypeptide heavy chains (H chains) and two polypeptide light chains (L chains) that are coupled by non-covalent disulfide bonds.

The term “immunological response” as used herein refers to the development in a subject of a humoral and/or a cellular immune response to an antigen present in the composition of interest. For purposes of the present disclosure, a “humoral immune response” refers to an immune response mediated by antibody molecules, while a “cellular immune response” is one mediated by T-lymphocytes and/or other white blood cells.

One aspect of cellular immunity involves an antigen-specific response by cytolytic T-cells (“CTL”s). CTLs have specificity for peptide antigens that are presented in association with proteins encoded by the major histocompatibility complex (MHC) and expressed on the surfaces of cells. CTLs help induce and promote the destruction of intracellular microbes or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells. Hence, an immunological response may include one or more of the following effects: the production of antibodies by B-cells; and/or the activation of suppressor T-cells and/or γδ T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

The term “immunogenic amount” as used herein refers to an amount capable of eliciting the production of antibodies directed against the virus in the host to which the vaccine has been administered.

The term “immunogenic carrier” as used herein refers to a composition enhancing the immunogenicity of the virosomes from any of the viruses discussed herein. Such carriers include, but are not limited to, proteins and polysaccharides, and microspheres formulated using, for example, a biodegradable polymer such as DL-lactide-coglycolide, liposomes, and bacterial cells and membranes. Protein carriers may be joined to the proteinases, or peptides derived therefrom, to form fusion proteins by recombinant or synthetic techniques or by chemical coupling. Useful carriers and ways of coupling such carriers to polypeptide antigens are known in the art.

The term “immunopotentiator,” as used herein, is intended to mean a substance that, when mixed with an immunogen, elicits a greater immune response than the immunogen alone. For example, an immunopotentiator can enhance immunogenicity and provide a superior immune response. An immunopotentiator can act, for example, by enhancing the expression of co-stimulators on macrophages and other antigen-presenting cells.

The terms “subject”, “individual”, or “patient” as used herein are used interchangeably and refer to an animal preferably a warm-blooded animal such as a mammal. Mammal includes without limitation any members of the Mammalia. A mammal, as a subject or patient in the present disclosure, can be from the family of Primates, Carnivora, Proboscidea, Perissodactyla, Artiodactyla, Rodentia, and Lagomorpha. In a particular embodiment, the mammal is a human. In other embodiments, animals can be treated; the animals can be vertebrates, including both birds and mammals. In aspects of the disclosure, the terms include domestic animals bred for food or as pets, including equines, bovines, sheep, poultry, fish, porcines, canines, felines, and zoo animals, goats, apes (e.g. gorilla or chimpanzee), and rodents such as rats and mice.

The term “pharmaceutically acceptable carrier” as used herein refers to a diluent, adjuvant, excipient, or vehicle with which a probe of the disclosure is administered and which is approved by a regulatory agency of the Federal or a state government or listed in the U.S. Pharmacopeia or other generally recognized pharmacopeia for use in animals, and more particularly in humans. Such pharmaceutical carriers can be liquids, such as water and oils, including those of petroleum, animal, vegetable or synthetic origin, such as peanut oil, soybean oil, mineral oil, sesame oil and the like. The pharmaceutical carriers can be saline, gum acacia, gelatin, starch paste, talc, keratin, colloidal silica, urea, and the like. When administered to a patient, the probe and pharmaceutically acceptable carriers can be sterile. Water is a useful carrier when the probe is administered intravenously. Saline solutions and aqueous dextrose and glycerol solutions can also be employed as liquid carriers, particularly for injectable solutions. Suitable pharmaceutical carriers also include excipients such as glucose, lactose, sucrose, glycerol monostearate, sodium chloride, glycerol, propylene, glycol, water, ethanol and the like. The present compositions, if desired, can also contain minor amounts of wetting or emulsifying agents, or pH buffering agents. The present compositions advantageously may take the form of solutions, emulsion, sustained-release formulations, or any other form suitable for use.

The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “vaccine” as used herein refers to an immunogenic amount of one or more virosomes, fragment(s), or subunit(s) thereof. Such vaccines can include one or more viral surface envelope glycoproteins and portions thereof, and adjuvant molecule and portions thereof on the surfaces of the virosomes, or in combination with another protein or other immunogen, such as one or more additional virus components naturally associated with viral particles or an epitopic peptide derived therefrom.

As used herein, the terms “effective amount” and “amount effective” refer to an amount that is sufficient to achieve the desired result or to have an effect on an undesired condition or prevention of a disease or condition or enhance and/or tune the immune system of the subject to the desirable responses for certain pathogens (e.g., virus). For example, a “therapeutically effective amount” refers to an amount that is sufficient to achieve the desired therapeutic result or to have an effect on undesired symptoms or prevention of a disease or condition and/or tune the immune system of the subject to the desirable responses for certain pathogens, but is generally insufficient to cause adverse side effects. The specific therapeutically effective dose level for any particular patient will depend upon a variety of factors including; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as infections and consequences thereof and/or tuning the immune system of the subject to the desirable responses for certain pathogens. The effect can be therapeutic in terms of a partial or complete cure of a disease, condition, symptom or adverse effect attributed to the disease, disorder, or condition. The term “treatment” as used herein can include any treatment of infections in a subject, particularly a human and can include any one or more of the following: (a) preventing the disease from occurring in a subject which may be predisposed to the disease or infection but has not yet been diagnosed as having it; (b) inhibiting the disease or infection, i.e., arresting its development; and (c) relieving the disease or infection i.e., mitigating or ameliorating the disease and/or its symptoms or conditions, (d) and/or tune the immune system of the subject to the desirable responses for certain pathogens. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. and/or tuning the immune system of the subject to the desirable responses for certain pathogens. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “therapeutic” can refer to treating, healing, and/or ameliorating a disease, disorder, condition, or side effect, or to decreasing in the rate of advancement of a disease, disorder, condition, or side effect and/or tuning the immune system of the subject to the desirable responses for certain pathogens.

The term “pharmaceutically acceptable” describes a material that is not biologically or otherwise undesirable, i.e., without causing an unacceptable level of undesirable biological effects or interacting in a deleterious manner.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

A saponin preparation isolated from the South American tree Quillaja saponaria Molina was first described by Dalsgaard et al. in 1974 (“Saponin adjuvants”, Archiv. fur die gesamte Virusforschung, Vol. 44, Springer Verlag, Berlin, p 243-254) to have adjuvant activity. Purified fragments of Quil A have been isolated by HPLC which retain adjuvant activity without the toxicity associated with Quil A (EP 0 362 278), for example QS7 and QS21 (also known as QA7 and QA21). QS-21 is a natural saponin derived from the bark of Quillaja saponaria Molina, that induces CD8+ cytotoxic T cells (CTLs), Th1 cells and a predominant IgG2a antibody response.

Saponins of the disclosure can be used at amounts between 1 and 100 μg per human dose of the adjuvant composition, at a level of about 50 μg, for example between 40 to 60 μg, suitably between 45 to 55 μg or between 49 and 51 μg or 50 μg. In some embodiments, a human dose of the adjuvant composition can comprise QS21 at a level of about 25 μg, for example between 20 to 30 μg, suitably between 21 to 29 μg or between 22 to 28 μg or between 28 and 27 μg or between 24 and 26 μg, or 25 μg.

When the adjuvant is to be combined with a liquid form of an antigenic composition, the adjuvant composition will be in a human dose suitable volume which is approximately half of the intended final volume of the human dose. For example a 500 μl volume of adjuvant for an intended final human dose of 1 μl, or a 250 μl volume for an intended final human dose of 0.5 ml. The adjuvant composition is diluted when combined with the antigen composition to provide the final human dose of vaccine. The final volume of such dose will of course vary dependent on the initial volume of the adjuvant composition and the volume of antigen composition added to the adjuvant composition. In an alternative embodiment, the aqueous adjuvant is used to reconstitute a lyophilized antigen composition. In this embodiment, the human dose suitable volume of the adjuvant composition is approximately equal to the final volume of the human dose. The liquid adjuvant composition is added to the vial containing the lyophilized antigen composition and used to reconstitute the lyophilized antigen composition.

### Abbreviations

IgG, immunoglobulin G; Th, T helper cells; CTL, cytotoxic T lymphocyte; rha, rhamnose; xyl, xylose; OVA, ovalbumin; NMM, N-methylmorpholine; HOBt, hydroxybenzotriazole; EDC HCl, 1-ethyl-3-(3-dimethylaminopropyl) carbodiimide; DCM, dichloromethane; MeCN, acetonitrile; THF, tetrahy-drofuran; rHagB, recombinant hemagglutinin B; s.c., subcutaneous; ESI-TOF, electrospray ionization time-of-flight mass spectrometry; ELISA, enzyme-linked immunosorbent assay;

### DISCUSSION

The present disclosure provides for compounds including modified saponin compounds, pharmaceutical compositions including modified saponin compounds, methods of use of the modified saponin compounds and the pharmaceutical compositions, methods of making modified saponin compounds, and the like. Compounds and pharmaceutical compositions of the present disclosure can be used in combination with one or more other therapeutic agents for treating viral infection and other diseases. For example, compounds and pharmaceutical compositions of the present disclosure can be employed in combination with other anti-viral agents to treat viral infection.

One aspect of the disclosure encompasses embodiments of a modified saponin having the formula:

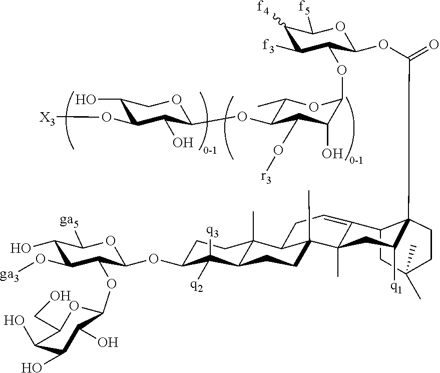

wherein:
q₁ can be H or OH;
q₂ and q₃ are each independently selected from CHO, CH₃, CH₂OH, H, COOH, a component of an acetal group, or an imine group;
f₃ and f₄ are each independently OH or an acetyl, or C3 and C4 of a fuocsyl unit wherein f₃, and f₄ can form a cyclic ketal ring or cyclic carbonate ester;
f₅ is selected from H, methyl group, —CH₂OH group, R₄—NR₅—C(O)—, R₄—O—C(O)—, or R₄—O—CH₂—;
wherein R₄ and R₅ are each independently a linear chain having the structure R₆[(CX₂)₀₋₂₀O₀₋₁(CY₂)₀₋₂₀]₀₋₂₀;
wherein X and Y are each independently H or a halogen atom;
wherein R₆ are COOR₇, C(O)NR₇R₈, NR₇R₈, or OR₇;
wherein R₇ are Ar[(CZ₂)₀₋₂₀O₀₋₁(CL₂)₀₋₂₀]₀₋₂₀, wherein R₈ is H or an alkyl;
wherein Z and L are each independently H or a halogen atom;
wherein Ar is a substituted or unsubstituted aromatic system;
ga₅ is selected from R₁₄—NR₁₅—C(O)—, R₁₄—O—C(O)—, or R₁₄—O—CH₂—;
wherein R₁₄ and R₁₅ are each independently a linear chain having the structure R₁₆[(CX1₂)₀₋₂₀O₀₋₁(CY1₂)₀₋₂₀]₀₋₂₀;
wherein X1 and Y1 are each independently H or a halogen atom;
wherein R₁₆ is COOR₁₇, C(O)NR₁₇R₁₈, NR₁₇R₁₈, or OR₁₇;
wherein R₁₇ is Ar2[(CZ1₂)₀₋₂₀O₀₋₁(CL1₂)₀₋₂₀]₀₋₂₀, wherein R₁₈ is H or an alkyl;
wherein Z1 and L1 are each independently H or a halogen atom;
wherein Ar2 is a substituted or unsubstituted aromatic group;
r3 is H, a monosaccharide, disaccharide, or a trisaccharide;
x3 is H, a monosaccharide (except xylose) or a disaccharide; and
ga3 is H, a monosaccharide or a disaccharide.

In some embodiments, the modified saponin can have the formula I:

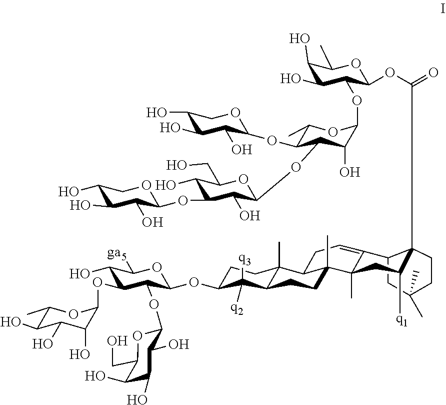

wherein:
q₁ is H or OH;
q₂ and q₃ are each independently selected from CHO, CH₃, CH₂OH, H, COOH, a component of an acetal group, or an imine group;
ga₅ is selected from R₁₄—NR₁₅—C(O)—, R₁₄—O—C(O)—, or R₁₄—O—CH₂—;
wherein R₁₄ and R₁₅ are each independently a linear chain having the structure R₁₆[(CX1₂)₀₋₂₀O₀₋₁(CY1₂)₀₋₂₀]₀₋₂₀;
wherein X1 and Y1 are each independently H or a halogen atom;
wherein R₁₆ is COOR₁₇, C(O)NR₁₇R₁₈, NR₁₇R₁₈, or OR₁₇;
wherein R₁₇ is Ar2[(CZ1₂)₀₋₂₀O₀₋₁(CL1₂)₀₋₂₀]₀₋₂₀, wherein R₁₈ is H or an alkyl;
wherein Z1 and L1 are each independently H or a halogen atom; and
wherein Ar2 is a substituted or unsubstituted aromatic group.

In other embodiments, the modified saponin can be selected from the group consisting of formulas 2D-2H:

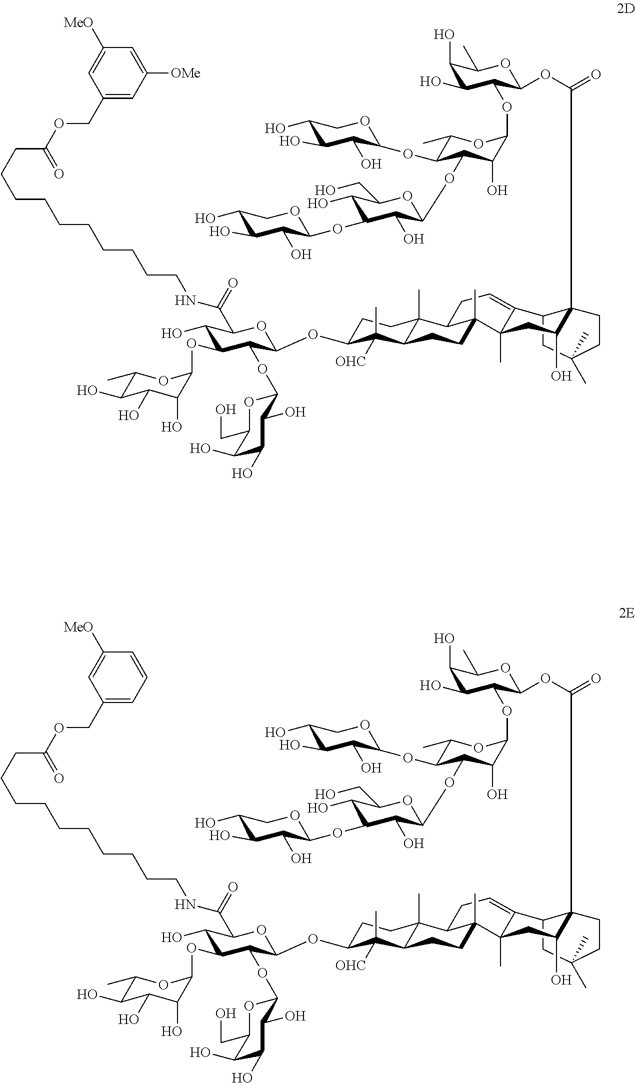

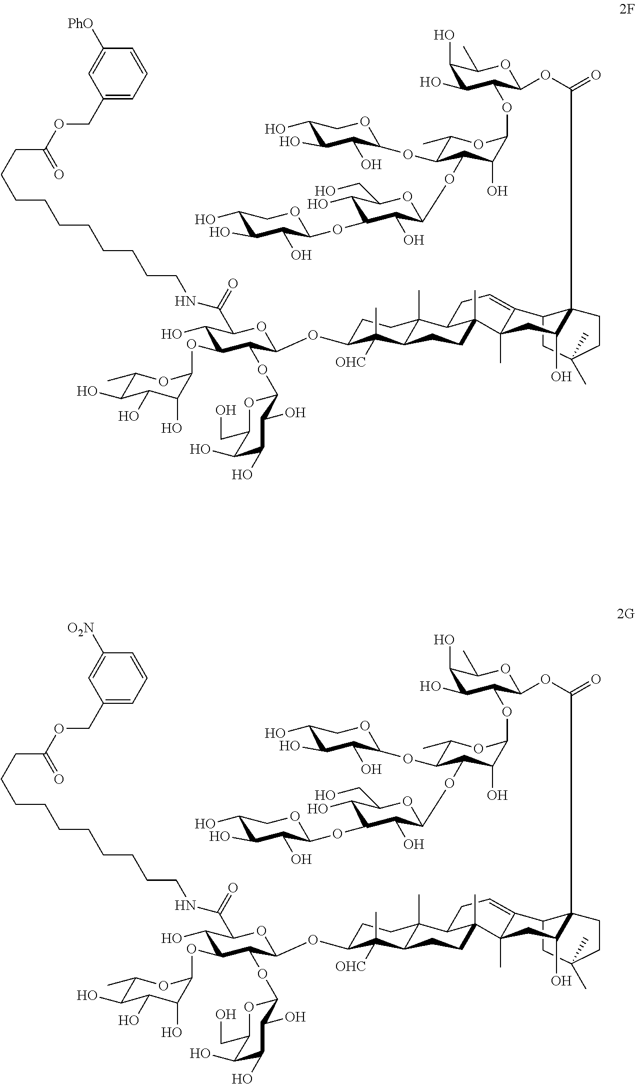

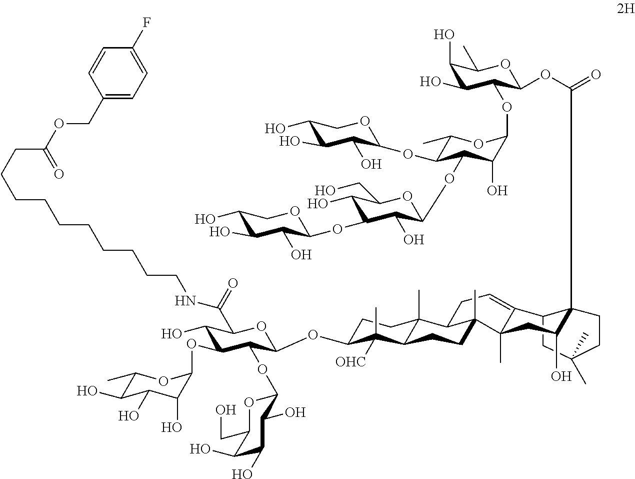

Another aspect of the disclosure encompasses embodiments of a pharmaceutical composition comprising a modified saponin having the formula:

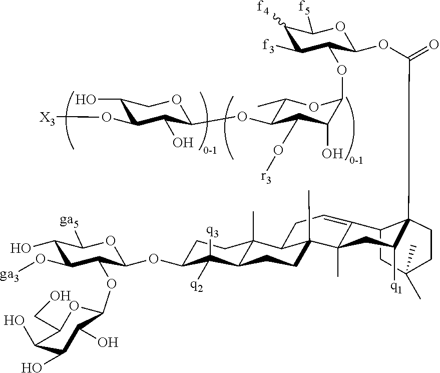

wherein:
q₁ can be H or OH;
q₂ and q₃ are each independently selected from CHO, CHs, CH₂OH, H, COOH, a component of an acetal group, or an imine group;
f₃ and f₄ are each independently OH or an acetyl, or C3 and C4 of a fuocsyl unit wherein f₃, and f₄ can form a cyclic ketal ring or cyclic carbonate ester;
f₅ is selected from H, methyl group, —CH₂OH group, R₄—NR₅—C(O)—, R₄—O—C(O)—, or R₄—O—CH₂—;
wherein R₄ and R₅ are each independently a linear chain having the structure R₆[(CX₂)₀₋₂₀O₀₋₁(CY₂)₀₋₂₀]₀₋₂₀;
wherein X and Y are each independently H or a halogen atom;
wherein R₆ are COOR₇, C(O)NR₇R₈, NR₇R₈, or OR₇;
wherein R₇ are Ar[(CZ₂)₀₋₂₀O₀₋₁(CL₂)₀₋₂₀]₀₋₂₀, wherein R₈ is H or an alkyl;
wherein Z and L are each independently H or a halogen atom;
wherein Ar is a substituted or unsubstituted aromatic system;
ga₅ is selected from R₁₄—NR₁₅—C(O)—, R₁₄—O—C(O)—, or R₁₄—O—CH₂—;
wherein R₁₄ and R₁₅ are each independently a linear chain having the structure R₁₆[(CX1₂)₀₋₂₀O₀₋₁(CY1₂)₀₋₂₀]₀₋₂₀;
wherein X1 and Y1 are each independently H or a halogen atom;
wherein R₁₆ is COOR₁₇, C(O)NR₁₇R₁₈, NR₁₇R₁₈, or OR₁₇;
wherein R₁₇ is Ar2[(CZ1₂)₀₋₂₀O₀₋₁(CL1₂)₀₋₂₀]₀₋₂₀, wherein R₁₈ is H or an alkyl;
wherein Z1 and L1 are each independently H or a halogen atom;
wherein Ar2 is a substituted or unsubstituted aromatic group;
r3 is H, a monosaccharide, disaccharide, or a trisaccharide;
x3 is H, a monosaccharide (except xylose) or a disaccharide; and
ga3 is H, a monosaccharide or a disaccharide.

Another aspect of the disclosure encompasses embodiments of a pharmaceutical composition comprising a modified saponin have the formula I:

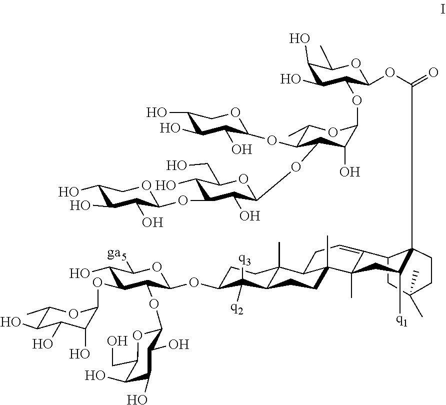

wherein:
q₁ is H or OH;
q₂ and q₃ are each independently selected from CHO, CH₃, CH₂OH, H, COOH, a component of an acetal group, or an imine group;
ga₅ is selected from R₁₄—NR₁₅—C(O)—, R₁₄—O—C(O)—, or R₁₄—O—CH₂—;
wherein R₁₄ and R₁₅ are each independently a linear chain having the structure R₁₆[(CX1₂)₀₋₂₀O₀₋₁(CY1₂)₀₋₂₀]₀₋₂₀;
wherein X1 and Y1 are each independently H or a halogen atom;
wherein R₁₈ is COOR₁₇, C(O)NR₁₇R₁₈, NR₁₇R₁₈, or OR₁₇;
wherein R₁₇ is Ar2[(CZ1₂)₀₋₂₀O₀₋₁(CL1₂)₀₋₂₀]₀₋₂₀, wherein R₁₈ is H or an alkyl;
wherein Z1 and L1 are each independently H or a halogen atom; and
wherein Ar2 is a substituted or unsubstituted aromatic group.

Another aspect of the disclosure encompasses embodiments of a pharmaceutical composition comprising a modified saponin having formulas 2D-2H:

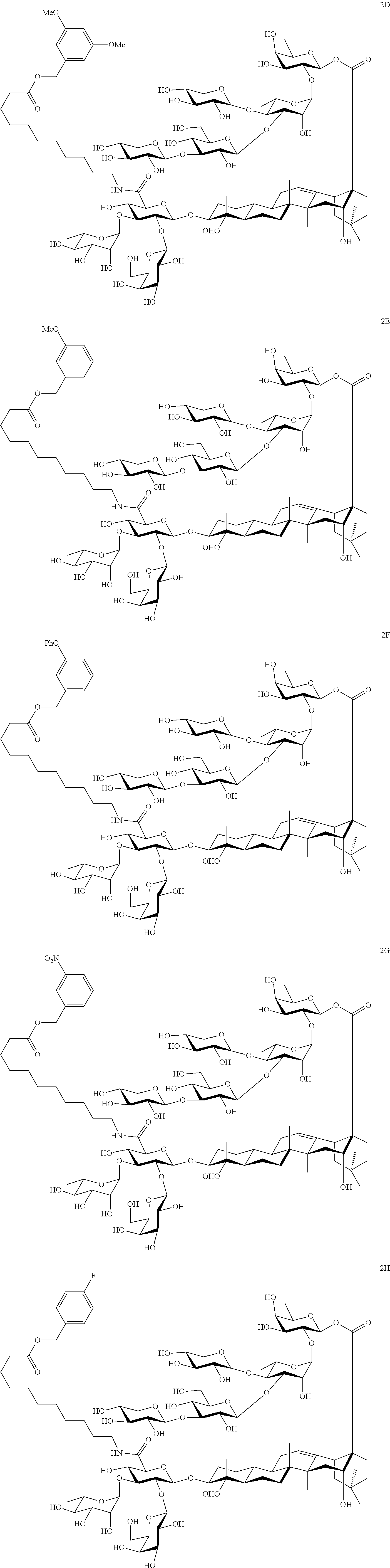

In some embodiments of this aspect of the disclosure, the pharmaceutical composition includes a therapeutically effective amount of a modified saponin to treat a condition (e.g., infection, disease, and the like) in a subject (e.g., animal or human subject). The pharmaceutical composition can further comprise at least one immunogen (e.g., in an immunogenic amount), an agent (e.g., a cancer agent), a pharmaceutically acceptable carrier, and the like. The present disclosure provides for administering to a subject in need thereof, a pharmaceutical composition including a therapeutically effective amount of a modified saponin or a pharmaceutically acceptable salt of the modified saponin of the active agent, and a pharmaceutically acceptable carrier.

The condition to be treated in a subject (e.g., mammal) in need of treatment can include those for which the vaccine is direct towards. The condition can be an infection (e.g., pathogen infection such as a viral infection such as coronavirus infection, HIV infection, influenza infection, hepatitis), disease such as cancer or the like. an infection or non-viral hyper-inflammation/immune hyper-activation condition.

In some embodiments of this aspect of the disclosure, the pharmaceutical composition can further comprise at least one cancer therapeutic agent. The at least one chemotherapeutic agent and the saponin derivative can be admixed in a pharmaceutically acceptable formulation or covalently linked to each other, and a pharmaceutically acceptable carrier. The cancer therapeutic agent can be present in a therapeutically effective amount to treat the condition.

Yet another aspect of the disclosure encompasses embodiments of a method of increasing the immunogenicity of an immunogen when administered to an animal or human subject, the method comprising the step of administering to the subject a vaccine comprising at least a pharmaceutical composition according to the disclosure.

Still yet another aspect of the disclosure encompasses embodiments of a synthetic route for the synthesis of a saponin derivative, the synthetic route comprising coupling a natural saponin with a functionalized side chain molecule, where the functionalized side chain comprises an amino group or hydroxyl group.

In some embodiments of this aspect of the disclosure, the natural saponin can be obtained from Momordica cochinchinensis Spreng.

In some embodiments of this aspect of the disclosure, the natural saponin can be coupled to the functionalized side chain molecule via an amide formation reaction or an ester formation reaction.

### Pharmaceutical Formulations and Routes of Administration

Embodiments of the present disclosure include the agent (e.g., the modified saponin) as identified herein and can be formulated with one or more pharmaceutically acceptable excipients, diluents, carriers and/or adjuvants. In addition, embodiments of the present disclosure include the agent formulated with one or more pharmaceutically acceptable auxiliary substances. In particular the agent can be formulated with one or more pharmaceutically acceptable excipients, diluents, carriers, and/or adjuvants to provide an embodiment of a composition of the present disclosure.

A wide variety of pharmaceutically acceptable excipients are known in the art. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In an embodiment of the present disclosure, the agent can be administered to the subject using any means capable of resulting in the desired effect. Thus, the agent can be incorporated into a variety of formulations for therapeutic administration. For example, the agent can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, the agent may be administered in the form of its pharmaceutically acceptable salts, or a subject active composition may be used alone or in appropriate association, as well as in combination, with other pharmaceutically active compounds. The following methods and excipients are merely exemplary and are in no way limiting.

For oral preparations, the agent can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Embodiments of the agent can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Embodiments of the agent can be utilized in aerosol formulation to be administered via inhalation. Embodiments of the agent can be formulated into pressurized acceptable propellants such as dichlorodifluoromethane, propane, nitrogen and the like.

Furthermore, embodiments of the agent can be made into suppositories by mixing with a variety of bases such as emulsifying bases or water-soluble bases. Embodiments of the agent can be administered rectally via a suppository. The suppository can include vehicles such as cocoa butter, carbowaxes and polyethylene glycols, which melt at body temperature, yet are solidified at room temperature.

Unit dosage forms for oral or rectal administration, such as syrups, elixirs, and suspensions, may be provided wherein each dosage unit, for example, teaspoonful, tablespoonful, tablet or suppository, contains a predetermined amount of the composition containing one or more compositions. Similarly, unit dosage forms for injection or intravenous administration may comprise the agent in a composition as a solution in sterile water, normal saline or another pharmaceutically acceptable carrier.

Embodiments of the agent can be formulated in an injectable composition in accordance with the disclosure. Typically, injectable compositions are prepared as liquid solutions or suspensions; solid forms suitable for solution in, or suspension in, liquid vehicles prior to injection may also be prepared. The preparation may also be emulsified or the active ingredient (triamino-pyridine derivative and/or the labeled triamino-pyridine derivative) encapsulated in liposome vehicles in accordance with the present disclosure.

In an embodiment, the agent can be formulated for delivery by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Mechanical or electromechanical infusion pumps can also be suitable for use with the present disclosure. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, delivery of the agent can be accomplished using any of a variety of refillable, pump systems. Pumps provide consistent, controlled release over time. In some embodiments, the agent can be in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to, a subdermal, subcutaneous, intramuscular, or other suitable site within a subject's body. Subcutaneous implantation sites are used in some embodiments because of convenience in implantation and removal of the drug delivery device.

Drug release devices suitable for use in the disclosure may be based on any of a variety of modes of operation. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump can also be suitable for use with the present disclosure. Examples of such devices include those described in, for example, U.S. Pat. Nos. 4,692,147; 4,360,019; 4,487,603; 4,360,019; 4,725,852, and the like. In general, a subject treatment method can be accomplished using any of a variety of refillable, non-exchangeable pump systems. Pumps and other convective systems are generally preferred due to their generally more consistent, controlled release over time. Osmotic pumps are used in some embodiments due to their combined advantages of more consistent controlled release and relatively small size (see, e.g., PCT published application no. WO 97/27840 and U.S. Pat. Nos. 5,985,305 and 5,728,396). Exemplary osmotically-driven devices suitable for use in the disclosure include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like.

In some embodiments, the drug delivery device is an implantable device. The drug delivery device can be implanted at any suitable implantation site using methods and devices well known in the art. As noted herein, an implantation site is a site within the body of a subject at which a drug delivery device is introduced and positioned. Implantation sites include, but are not necessarily limited to a subdermal, subcutaneous, intramuscular, or other suitable site within a subject's body.

In some embodiments, the agent can be delivered using an implantable drug delivery system, e.g., a system that is programmable to provide for administration of the agent. Exemplary programmable, implantable systems include implantable infusion pumps. Exemplary implantable infusion pumps, or devices useful in connection with such pumps, are described in, for example, U.S. Pat. Nos. 4,350,155; 5,443,450; 5,814,019; 5,976,109; 6,017,328; 6,171,276; 6,241,704; 6,464,687; 6,475,180; and 6,512,954. A further exemplary device that can be adapted for the present disclosure is the Synchromed infusion pump (Medtronic).

Suitable excipient vehicles for the agent are, for example, water, saline, dextrose, glycerol, ethanol, or the like, and combinations thereof. In addition, if desired, the vehicle may contain minor amounts of auxiliary substances such as wetting or emulsifying agents or pH buffering agents. Methods of preparing such dosage forms are known, or will be apparent upon consideration of this disclosure, to those skilled in the art. See, e.g., Remington's Pharmaceutical Sciences, Mack Publishing Company, Easton, Pennsylvania, 17th edition, 1985. The composition or formulation to be administered will, in any event, contain a quantity of the agent adequate to achieve the desired state in the subject being treated.

Compositions of the present disclosure can include those that comprise a sustained-release or controlled release matrix. In addition, embodiments of the present disclosure can be used in conjunction with other treatments that use sustained-release formulations. As used herein, a sustained-release matrix is a matrix made of materials, usually polymers, which are degradable by enzymatic or acid-based hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxcylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. Illustrative biodegradable matrices include a polylactide matrix, a polyglycolide matrix, and a polylactide co-glycolide (co-polymers of lactic acid and glycolic acid) matrix.

In another embodiment, the pharmaceutical composition of the present disclosure (as well as combination compositions) can be delivered in a controlled release system. For example, the agent may be administered using intravenous infusion, an implantable osmotic pump, a transdermal patch, liposomes, or other modes of administration. In one embodiment, a pump may be used (Sefton (1987). CRC Crit. Ref. Biomed. Eng. 14:201; Buchwald et al. (1980). Surgery 88:507; Saudek et al. (1989). N. Engl. J. Med. 321:574). In another embodiment, polymeric materials are used. In yet another embodiment a controlled release system is placed in proximity of the therapeutic target thus requiring only a fraction of the systemic dose. In yet another embodiment, a controlled release system is placed in proximity of the therapeutic target, thus requiring only a fraction of the systemic. Other controlled release systems are discussed in the review by Langer (1990). Science 249:1527-1533.

In another embodiment, the compositions of the present disclosure (as well as combination compositions separately or together) include those formed by impregnation of the agent described herein into absorptive materials, such as sutures, bandages, and gauze, or coated onto the surface of solid phase materials, such as surgical staples, zippers and catheters to deliver the compositions. Other delivery systems of this type will be readily apparent to those skilled in the art in view of the instant disclosure.

### Dosages

Embodiments of the agent (e.g., the modified saponin) can be administered to a subject in one or more doses. Those of skill will readily appreciate that dose levels can vary as a function of the specific the agent administered, the severity of the symptoms and the susceptibility of the subject to side effects. Preferred dosages for a given compound are readily determinable by those of skill in the art by a variety of means.

In an embodiment, multiple doses of the agent are administered. The frequency of administration of the agent can vary depending on any of a variety of factors, e.g., severity of the symptoms, and the like. For example, in an embodiment, the agent can be administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid). As discussed above, in an embodiment, the agent is administered continuously.

The duration of administration of the agent, e.g., the period of time over the agent is administered, can vary, depending on any of a variety of factors, e.g., patient response, etc. For example, the agent in combination or separately, can be administered over a period of time of about one day to one week, about two weeks to four weeks, about one month to two months, about two months to four months, about four months to six months, about six months to eight months, about eight months to 1 year, about 1 year to 2 years, or about 2 years to 4 years, or more.

Dosage at concentrations as high as 60 micrograms/kilograms that are non-toxic. Also lower concentrations, such as 1˜4 micrograms/kilogram, show biological activity in in vivo systems. The concentration in in vitro established at 10⁻⁹-10⁻⁶ M are active and this concentration is expected to be achieved in the cell environment. (See Slominski A T, Janjetovic Z, Fuller B E, Zmijewski M A, Tuckey R C, et al. (2010) Products of vitamin D3 or 7-dehydrocholesterol metabolism by cytochrome P450scc show anti-leukemia effects, having low or absent calcemic activity. PLOS ONE 5(3): e990; Slominski A T, Kim T-K., Janjetovic Z, Tuckey R C, Bieniek, R, Yue Y, Li W, Chen J, Miller D, Chen T, Holick M (2011) 20-hydroxyvitamin D2 is a non-calcemic analog of vitamin D with potent antiproliferative and prodifferentiation activities in normal and malignant cells. Am J Physiol: Cell Physiol 300:C526-C541; Wang J, Slominski A T, Tuckey R C, Janjetovic Z, Kulkarni A, Chen J, Postlethwaite A, Miller D, Li W (2012) 20-Hydroxylvitamin D₃ possesses high efficacy against proliferation of cancer cells while being non-toxic. Anticancer Res 32: 739-746; Slominski A, Janjetovic Z, Tuckey R C, Nguyen M N, Bhattacharya K G, Wang J, Li W, Jiao Y, Gu W, Brown M, Postlethwaite A E (2013) 20-hydroxyvitamin D3, noncalcemic product of CYP11A1 action on vitamin D3, exhibits potent antifibrogenic activity in vivo. J Clin Endocrinol Metab 98, E298-E30; Chen, J., J. Wang, T. Kim, E. Tieu, E. Tamg, Lin Z, D. Kovacic, D. Miller, A. Postlethwaite, R. Tuckey, A. Slominski and W. Li (2014). Novel Vitamin D Analogs as Potential Therapeutics: The Metabolism, Toxicity Profiling, and Antiproliferative Activity. Anticancer Res 34:2153-2163.)

In an aspect, the dosage for administering to a subject (e.g., a mammal such as a human) having a condition (e.g., COVID-19) of any single agent the present disclosure is about 2 to 60 micrograms/kilogram or a combination of agents, each agent can be about 2 to 60 micrograms/kilogram.

### Routes of Administration

Embodiments of the present disclosure provide methods and compositions for the administration of the agent (e.g., the modified saponin) to a subject (e.g., a human) using any available method and route suitable for drug delivery, including in vivo and ex vivo methods, as well as systemic and localized routes of administration.

Routes of administration include intranasal, intramuscular, intratracheal, subcutaneous, intradermal, topical application, intravenous, rectal, nasal, oral, and other enteral and parenteral routes of administration. Routes of administration may be combined, if desired, or adjusted depending upon the agent and/or the desired effect. An agent can be administered in a single dose or in multiple doses.

Embodiments of the agent can be administered to a subject using available conventional methods and routes suitable for delivery of conventional drugs, including systemic or localized routes. In general, routes of administration contemplated by the disclosure include, but are not limited to, enteral, parenteral, or inhalational routes.

Parenteral routes of administration other than inhalation administration include, but are not limited to, topical, transdermal, subcutaneous, intramuscular, intraorbital, intracapsular, intraspinal, intrasternal, and intravenous routes, i.e., any route of administration other than through the alimentary canal. Parenteral administration can be conducted to effect systemic or local delivery of the agent. Where systemic delivery is desired, administration typically involves invasive or systemically absorbed topical or mucosal administration of pharmaceutical preparations.

In an embodiment, the agent can also be delivered to the subject by enteral administration. Enteral routes of administration include, but are not limited to, oral and rectal (e.g., using a suppository) delivery.

Methods of administration of the agent through the skin or mucosa include, but are not limited to, topical application of a suitable pharmaceutical preparation, transdermal transmission, injection and epidermal administration. For transdermal transmission, absorption promoters or iontophoresis are suitable methods. Iontophoretic transmission may be accomplished using commercially available “patches” that deliver their product continuously via electric pulses through unbroken skin for periods of several days or more.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

### Example 1

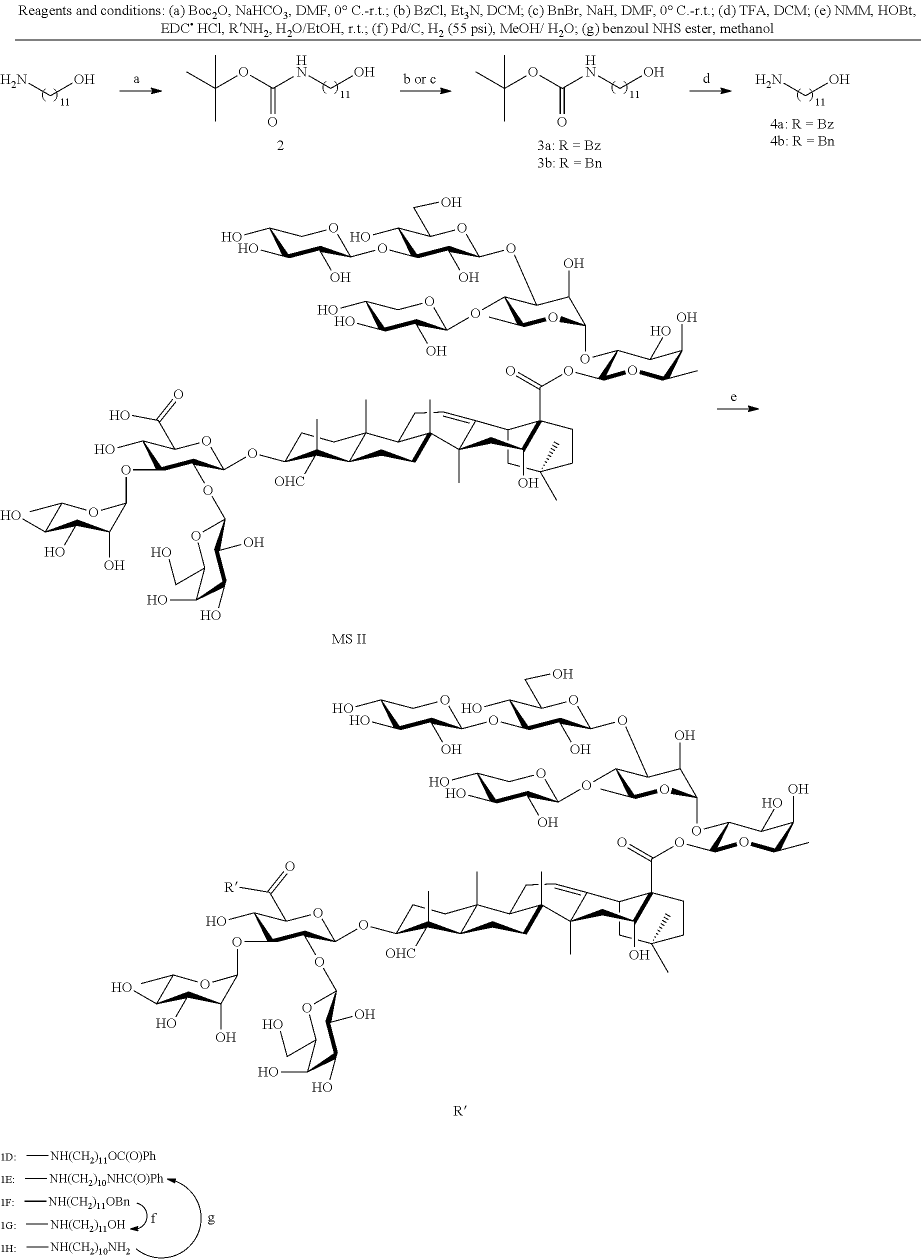

### Synthesis of 2.

(BOC)₂O (33 mg, 0.15 mmol) was added to 11-amino-1-undecanol (20 mg, 0.1 mmol) stirring in N,N-dimethylformamide (0.5 mL) and NaHCO₃ (5 mg, 20%, v/v) at 0° C. The reaction mixture was stirred for overnight at room temperature. After that, the reaction mixture was filtered through the column chromatography packed with silica gel and ethyl acetate to obtain 2 which was directly used in the synthesis of 3a and 3b.

### Synthesis of 3a.

Benzoyl chloride (230 μl, 2 mmol) was added to the intermediate 2 ((30 mg, 0.1 mmol) stirring in the mixture of dichloromethane (2.25 mL) and triethylamine (1.39 mL, 10 mmol) at room temperature. The mixture was stirred for 48 hrs and then concentrated with rotary evaporator to be extracted with EtOAc. The organic layer was washed with NaHCO3 for three times and dried with Na₂SO₄. The collected organic layer was then purified with column chromatography on silica gel (eluted with petroleum ether and ethyl acetate gradient) to afford 3b (15 mg, 38%). Rf (PE/EtOAc=9:1)=0.49. ¹H NMR (700 MHZ, CDCl3): δ 8.04 (dd, J=8.0, 1.0 Hz, 2H), 7.56 (tt, J=7.4, 1.2 Hz, 1H), 7.43 (td, J=8.0 Hz, 2H), 4.53 (b, 1H), 4.31 (t, J=6.7 Hz, 2H), 3.10 (b, 2H), 1.76 (quint, J=7.2 Hz, 2H), 1.44 (b, 11H), 1.35 (quint, J=7.2 Hz, 2H), 1.27 (b, 12H); ¹³C NMR (176 MHZ, CD3OD): δ 166.6, 155.9, 132.7, 130.5, 129.5, 128.3, 65.1, 40.6, 30.0, 29.7, 29.5, 29.4, 29.2, 28.7, 28.4, 26.8, 26.0; HRMS (ESI-TOF) m/z: [M+H-Boc]⁺ calcd for C₁₈H₃₀NO₂, 292.2277; found, 292.2269.

### Synthesis of 3b.

Benzyl bromide (25 μl, 0.21 mmol) was added to the mixture of sodium hydride (5 mg, 0.21 mmol) and the intermediate 2 (50 mg, 0.175 mmol), which was already stirred in an ice bath for 15 mins in dry N, N-dimethylformamide (1.5 mL). The mixture was stirred at room temperature for overnight. Then the mixture was extracted using EtOAc and washed with deionized water for three times. The organic layer that was collected was dried over the sodium sulfate. After that, the organic layer was concentrated through rotary evaporator and then the mixture was purified with column chromatography on silica gel (eluted with petroleum ether and ethyl acetate gradient) to produce the desired compound 3b (29 mg, 50%). R_{f} (PE/EtOAc=9:1)=0.71 ¹H NMR (700 MHZ, CD₃OD) (characteristic protons): δ 7.35-7.32 (m, 4H), 7.28 (m, 1H), 5.30 (s, 1H), 4.50 (s, 2H), 3.46 (t, J=6.7, Hz, 2H); 3.10 (q, J=6.1 Hz, 2H); 1.61 (quint, J=7.1 Hz, 2H); 1.44 (s, 11H), 1.35 (quint, J=7.0 Hz, 2H), 1.31-1.22 (m, 12H); ¹³C NMR (176 MHz, CD₃OD): δ 155.9, 138.7, 128.3, 127.6, 127.4, 72.8, 70.5, 53.4, 40.6, 30.0, 29.73, 29.67, 29.52, 29.50, 29.47, 29.44, 29.3, 28.4, 26.8, 26.2; HRMS (ESI-TOF) m/z: [M+H-Boc]⁺ calcd for C₁₈H₃₂NO, 278.2484; found, 278.2480.

### Synthesis of 4a.

Trifluoroacetic acid (85 μl, 30%, v/v) was added to the mixture of 3a (20 mg, 0.07 mmol) and dry dichloromethane (1 mL) to deprotect the boc protecting group. The mixture was stirred for 3 hrs and quenched with Na₂CO₃. The mixture was concentrated through the rotary evaporator to afford the product (6.5 mg, 33%)

### Synthesis of 4b.

Trifluoroacetic acid (25 μl, 30% (v/v)) was added to the mixture of 3b (6 mg, 0.016 mmol) and dry dichloromethane (0.5 mL). The reaction mixture was concentrated and washed with Na₂CO₃. It was concentrated through the rotary evaporator to afford the product 4b (12 mg, 82%).

### General Procedure of Derivatizing MS II (Unoptimized):

To MS II in ethanol/water were added the side chain, N-methylmorpholine (NMM), hydroxylbenzotriazole (HOBt) and 1-ethyl-3-(3-dimethylaminopropyl) carbodiimide hydrochloride (EDC·HCl) at room temperature. The reaction mixture was stirred for 3 days and then was directly purified with RP HPLC by using a semi-Prep C₁₈, 250×10 mm, 5 micron column and H2O/MeCN gradients (90%-10% H₂O over 45 minutes with a 3 mL/min flow rate). The product fraction was concentrated on a rotary evaporator at room temperature to remove MeCN, and the remaining water was then removed on a lyophilizer to provide the derivative as a white solid.

For 1D, (12.4 mg, 35%): ¹H NMR (500 MHZ, CD₃OD) (characteristic protons): δ 9.50 (s, 1H), 8.03 (dd, J=8.4, 1.3 Hz, 2H), 7.87 (t, J=5.7 Hz, 1H), 7.63 (tt, J=7.4, 1.3 Hz, 1H), 7.50 (t, J=7.8 Hz, 2H), 5.43 (d, J=1.5 Hz, 1H), 5.33 (b, 1H), 5.23 (d, J=8.2 Hz, 1H), 5.04 (d, J=1.4 Hz, 1H), 4.73 (d, J=7.9 Hz, 1H), 4.56 (d, J=7.8 Hz, 1H), 4.52 (b, 1H), 4.50-4.40 (m, 3H), 4.35 (t, J=6.6 Hz, 2H), 4.24 (dd, J=2.8, 1.9 Hz, 1H), 4.06-4.01 (m, 2H), 2.30 (t, J=13.6 Hz, 1H), 1.42 (s, 3H), 1.19 (s, 3H), 1.03 (s, 3H), 0.94 (s, 3H), 0.88 (s, 3H), 0.80 (s, 3H); ¹³C NMR (176.0 MHz, CD₃OD): δ 209.6, 175.5, 169.8, 166.7, 143.6, 132.8, 130.2, 129.1, 128.2, 121.5, 104.6, 103.7, 103.5, 102.8, 101.9, 99.3, 94.0, 87.4, 84.6, 84.5, 82.2, 77.3, 76.8, 76.5, 76.0, 75.4, 75.1, 74.2, 74.0, 73.6, 73.3, 73.0, 72.4, 72.3, 71.5, 70.8, 70.6, 70.5, 70.0, 68.0, 67.4, 65.7, 64.9, 60.8, 60.6, 54.8, 41.5, 41.0, 39.7, 38.7, 38.0, 35.7, 35.2, 32.0, 30.5, 29.9, 29.5, 29.3, 29.2, 29.0, 28.9, 28.4, 26.6, 26.0, 25.8, 24.6, 23.3, 23.0, 20.0, 17.0, 16.5, 16.4, 15.1, 9.6; HRMS (ESI-TOF) m/z: [M+H]⁺ calcd for C₉₄H₁₄₈NO₄₂, 1962.9476; found, 1962.9467.

For 1E (8.6 mg, 56% from 1H with benzoyl N-hydroxysuccinimide ester): ¹HNMR (600 MHZ, CD₃OD) (characteristic protons): δ 9.49 (s, 1H), 7.83 (d, J=7.4 Hz, 2H), 7.54 (tt, J=7.4, 1.2 Hz, 1H), 7.48 (d, J=7.4 Hz, 2H), 5.43 (d, J=1.6 Hz, 1H), 5.33 (t, J=3.7 Hz, 1H), 5.23 (d, J=8.1 Hz, 1H), 5.04 (d, J=1.7 Hz, 1H), 4.74 (d, J=7.9 Hz, 1H), 4.56 (d, J=7.7 Hz, 1H), 4.53 (b, 1H), 4.50-4.46 (m, 2H), 4.45 (d, J=7.7 Hz, 1H), 4.25 (dd, J=2.9, 2.2 Hz, 1H), 4.06-4.01 (m, 2H), 3.18 (t, J=11.3 Hz, 1H), 3.14 (dd, J=9.4, 8.1 Hz, 1H), 2.90 (dd, J=14.6, 4.5 Hz, 1H), 2.30 (t, J=13.7 Hz, 1H), 1.18 (s, 3H), 1.03 (s, 3H), 0.93 (s, 3H), 0.88 (s, 3H), 0.81 (s, 3H); ¹³C NMR (150.9 MHZ, CD₃OD): δ 209.6, 175.6, 169.8, 168.8, 143.5, 134.5, 131.1, 128.2, 126.8, 121.6, 104.7, 103.8, 103.5, 102.8, 102.7, 101.9, 99.3, 94.0, 87.4, 84.6, 84.5, 82.2, 77.3, 76.8, 76.4, 76.0, 75.4, 75.1, 74.2, 74.0, 73.6, 73.4, 73.0, 72.4, 72.3, 71.6, 71.5, 70.8, 70.7, 70.5, 70.1, 70.0, 69.6, 69.2, 69.1, 68.0, 67.4, 65.7, 60.8, 54.8, 48.5, 46.9, 46.6, 41.5, 41.0, 39.7, 38.7, 38.0, 35.7, 35.2, 32.8, 31.9, 30.5, 29.9, 29.4, 29.3, 29.1, 28.9, 26.7, 26.5, 25.9, 24.5, 23.3, 23.1, 20.0, 17.0, 16.5, 16.4, 15.1, 9.6; HRMS (ESI-TOF) m/z: [M+H]⁺ calcd for C₉₃H₁₄₇N₂O₄₁, 1947.9479; found, 1947.9386.

For 1F (8.3 mg, 36%): ¹HNMR (600 MHZ, CD₃OD) (characteristic protons): δ 9.50 (s, 1H), 7.37-7.34 (m, 4H), 7.30 (m, 1H), 5.43 (d, J=1.5 Hz, 1H), 5.33 (t, J=3.3 Hz, 1H), 5.30 (d, J=8.3 Hz, 1H), 5.04 (d, J=1.6 Hz, 1H), 4.74 (d, J=8.1 Hz, 1H), 4.56 (d, J=8.0 Hz, 1H), 4.25 (dd, J=2.9, 1.7 Hz, 1H), 2.91 (dd, J=14.2, 4.5 Hz, 1H), 2.30 (t, J=13.6 Hz, 1H), 1.19 (s, 3H), 1.03 (s, 3H), 0.94 (s, 3H), 0.88 (s, 3H), 0.81 (s, 3H); ¹³C NMR (176.0 MHZ, CD₃OD): δ 209.6, 175.5, 169.7, 143.6, 138.5, 121.5, 104.7, 103.8, 103.5, 102.8, 102.7, 101.9, 99.3, 94.0, 87.4, 84.6, 84.5, 82.2, 77.2, 76.8, 76.4, 76.0, 75.4, 75.1, 74.2, 74.0, 73.6, 73.3, 73.0, 72.5, 72.4, 72.3, 71.5, 71.4, 70.8, 70.7, 70.5, 70.0, 69.6, 69.2, 69.1, 68.0, 67.4, 65.7, 60.8, 60.6, 54.8, 48.4, 41.5, 41.0, 39.7, 38.7, 35.7, 35.2, 32.7, 32.0, 29.9, 29.5, 29.4, 29.3, 29.2, 28.9, 26.6, 25.9, 23.3, 17.0, 16.5, 16.4, 15.1, 9.6; HRMS (ESITOF) m/z: [M+H]⁺ calcd for C₉₄H₁₅₀NO₄₁, 1948.9383; found, 1948.9684.

For 1G (7.4 mg, 67% from 1F): ¹HNMR (600 MHZ, CD₃OD) (characteristic protons): δ 9.49 (s, 1H), 5.42 (d, J=1.6 Hz, 1H), 5.32 (t, J=3.5 Hz, 1H), 5.22 (d, J=8.3 Hz, 1H), 5.03 (d, J=1.4 Hz, 1H), 4.73 (d, J=7.8 Hz, 1H), 4.55 (d, J=7.8 Hz, 1H), 4.52 (b, 1H), 4.44 (d, J=7.6 Hz, 1H), 4.23 (dd, J=2.8, 1.8 Hz, 1H), 3.17 (t, J=10.9 Hz, 1H), 3.12 (dd, J=8.2, 8.1 Hz, 1H), 2.90 (dd, J=14.0, 4.4 Hz, 1H), 2.30 (t, J=13.6 Hz, 1H), 1.42 (s, 3H), 1.19 (s, 3H), 1.03 (s, 3H), 0.95 (s, 3H), 0.88 (s, 3H), 0.80 (s, 3H); ¹³C NMR (213.8 MHz, CD₃OD): δ 209.6, 175.6, 169.8, 143.5, 121.5, 104.7, 103.8, 103.6, 102.8, 102.7, 101.9, 99.3, 94.0, 87.4, 84.6, 84.5, 82.1, 77.2, 76.9, 76.4, 76.0, 75.4, 75.1, 74.2, 74.0, 73.6, 73.3, 73.0, 72.4, 72.3, 71.7, 71.5, 70.8, 70.7, 70.5, 70.1, 70.0, 69.6, 69.2, 69.1, 68.0, 67.4, 65.7, 61.6, 60.8, 60.6, 54.8, 46.6, 41.5, 41.0, 39.7, 38.7, 38.0, 35.7, 35.2, 32.7, 32.3, 32.0, 31.7, 30.5, 29.9, 29.5, 29.4, 29.3, 29.2, 29.1, 28.9, 26.5, 25.9, 25.6, 24.5, 23.3, 23.1, 22.3, 20.0, 17.0, 16.5, 16.4, 15.1, 9.6; HRMS (ESI-TOF) m/z: [M+H]+ calcd for C₈₇H₁₄₄NO₄₁, 1858.9214; found, 1858.9211.

For 1H (20.4 mg, 27%): ¹H NMR (600 MHZ, CD₃OD) (characteristic protons): δ 9.50 (s, 1H), 5.42 (d, J=1.1 Hz, 1H), 5.33 (t, J=3.3 Hz, 1H), 5.24 (d, J=8.4 Hz, 1H), 5.04 (d, J=1.4 Hz, 1H), 4.74 (d, J=8.0 Hz, 1H), 4.56 (d, J=8.0 Hz, 1H), 4.53 (b, 1H), 4.50-4.46 (m, 2H), 4.45 (d, J=7.7 Hz, 1H), 4.25 (dd, J=3.1, 1.9 Hz, 1H), 4.05-4.01 (m, 2H), 3.18 (t, J=11.1 Hz, 1H), 3.14 (dd, J=9.4, 8.1 Hz, 1H), 2.31 (t, J=13.6 Hz, 1H), 1.20 (s, 3H), 1.03 (s, 3H), 0.96 (s, 3H), 0.89 (s, 3H), 0.81 (s, 3H); ¹³C NMR (150.9 MHZ, CD₃OD): δ 209.6, 175.6, 169.8, 143.6, 121.5, 104.7, 103.8, 103.5, 102.8, 101.7, 101.9, 99.3, 94.0, 87.4, 84.6, 82.1, 77.3, 76.9, 76.4, 76.0, 75.4, 75.1, 74.3, 74.0, 73.6, 73.3, 73.0, 72.4, 72.3, 71.7, 71.5, 70.8, 70.6, 70.5, 70.1, 70.0, 69.6, 69.2, 69.1, 68.0, 67.4, 65.7, 60.8, 60.7, 54.8, 48.5, 46.6, 41.5, 41.0, 39.7, 39.4, 38.7, 37.9, 35.7, 35.2, 32.7, 32.0, 30.5, 29.9, 29.2, 29.0, 28.9, 28.8, 27.2, 26.4, 26.0, 25.9, 24.5, 23.2, 23.1, 20.0, 17.0, 16.5, 16.4, 15.1, 15.0, 9.6; HRMS (ESI-TOF) m/z: [M+H]⁺ calcd for C₈₆H₁₄₃N₂O₄₀, 1843.9217; found, 1843.9216.

### Example 2

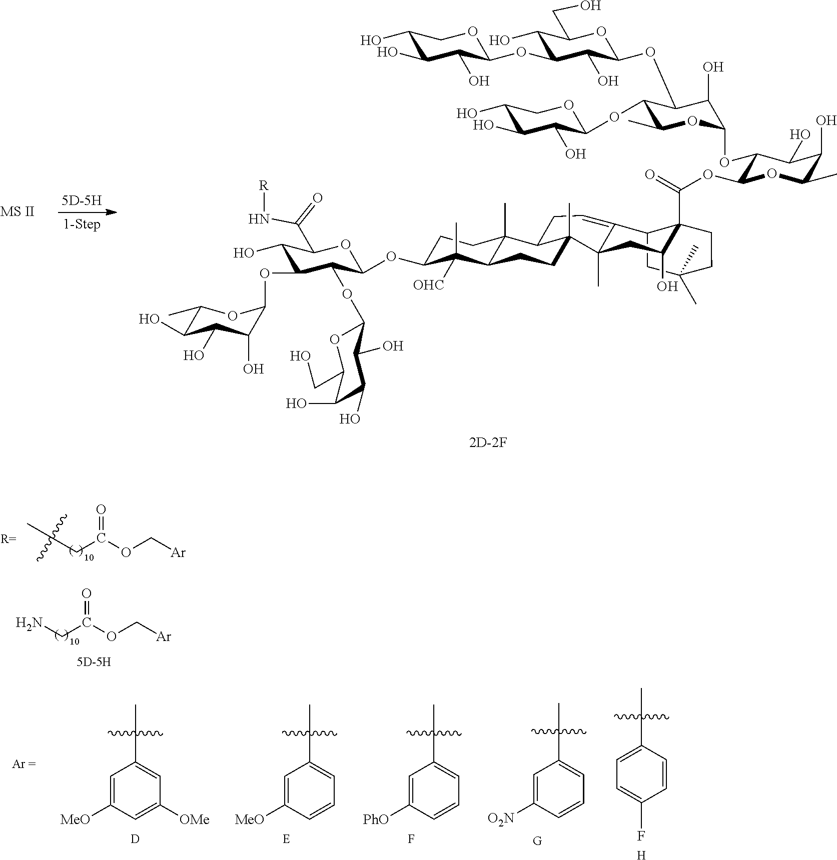

### General Procedure of Preparing Side Chains 5D-5H (Unoptimized):

A mixture of 11-aminoundecanoic acid (50 mg, 0.25 mmol) and thionyl chloride (36 μL, 0.5 mmol) stirred at room temperature first and then was heated up to 70° C. in an oil bath and stirred for about 1 hr. To the reaction mixture, a substituted benzyl alcohol (0.5 mmol) was added and stirred overnight. The mixture was purified with column chromatography on silica (eluted with DCM/MeOH gradient) to produce the desired side chain.

### Synthesis of 5D.

3,5-dimethoxybenzylalcohol (84.0 mg, 0.5 mmol) was used and purification with column chromatography on silica (eluted with DCM/MeOH gradient) produced 5D (41.0 mg, 47%). R_{f} (DCM/MeOH 9:1) 0.43; ¹H NMR (700 MHZ, CD₃OD): δ 6.48 (d, J=2.2 Hz, 2H), 6.40 (t, J=2.2 Hz, 1H), 5.03 (s, 2H), 3.77 (s, 6H), 2.97 (t, J=7.9 Hz, 2H), 2.34 (t, J=7.6 Hz, 2H), 1.75 (quint, J=7.8 Hz, 2H), 1.63 (quint, J=7.8 Hz, 2H), 1.35 (quint, J=7.3 Hz, 2H), 1.32-1.22 (m, 10H); ¹³C NMR (176.0 MHZ, CD₃OD): δ 173.5, 160.9, 138.4, 105.9, 100.1, 65.9, 55.3, 40.0, 34.3, 29.4, 29.3, 29.2, 29.1, 29.0, 27.6, 26.6, 24.9; HRMS (ESI-TOF) m/z: [M+H]⁺ calcd for C₂₀H₃₄NO₄, 352.2488; found, 352.2486.

### Synthesis of 5E.

3-methoxybenzylalcohol (63.0 mg, 0.5 mmol) and purification with column chromatography on silica (eluted with DCM/MeOH gradient) produced 5E (65.0 mg, 81%). R_{f} (DCM/MeOH 9:1) 0.56; ¹H NMR (700 MHZ, CD₃OD): δ 7.26 (d, J=7.9 Hz, 1H), 6.92 (d, J=7.3 Hz, 1H), 6.88 (s, 1H), 6.85 (dd, J=7.7, 2.1 Hz, 1H), 5.08 (s, 2H), 3.80 (s, 3H), 2.97 (t, J=7.7 Hz, 2H), 2.34 (t, J=7.9 Hz, 2H), 1.76 (quint, J=7.7 Hz, 2H), 1.63 (quint, J=7.3 Hz, 2H), 1.37 (quint, J=7.3 Hz, 2H), 1.33-1.23 (m, 10H); ¹³C NMR (176.0 MHZ, CD₃OD): δ 173.6, 159.8, 137.7, 129.6, 120.3, 113.6, 65.9, 55.2, 40.0, 34.3, 29.6, 29.4, 29.3, 29.1, 28.9, 27.7, 26.5, 24.9; HRMS (ESI-TOF) m/z: [M+H]⁺ calcd for C₁₉H₃₂NO₃, 322.2382; found, 322.2380.

### Synthesis of 5F:

3-Phenoxyphenylmethanol (87.0 μL, 0.5 mmol) and purification with column chromatography on silica (eluted with DCM/MeOH gradient) produced 5F (50.0 mg, 52%). R_{f} (DCM/MeOH 9:1) 0.43; ¹H NMR (700 MHZ, CD₃OD) δ 7.32 (t, J=17.0 Hz, 2H), 7.29 (t, J=7.9 Hz, 1H), 7.10 (t, J=7.4 Hz, 1H), 7.06 (d, J=7.7 Hz, 1H), 7.00-6.98 (m, 3H), 6.93 (dd, J=8.2, 2.2 Hz, 1H), 5.07 (s, 3H), 2.97 (t, J=7.9 Hz, 2H), 2.33 (t, J=7.5 Hz, 2H), 1.76 (quint, J=7.7 Hz, 2H), 1.61 (quint, J=7.4 Hz, 2H), 1.37 (quint, J=7.4 Hz, 2H), 1.33-1.23 (m, 10H); ¹³C NMR (176.0 MHZ, CD₃OD): δ 173.4, 157.5, 157.0, 138.2, 129.8, 129.6, 123.4, 122.6, 119.0, 118.3, 118.2, 65.5, 40.0, 34.3, 29.7, 29.3, 29.2, 29.1, 29.0, 27.7, 26.6, 24.9; HRMS (ESI-TOF) m/z: [M+H]⁺ calcd for C₂₄H₃₄NO₃, 384.2539; found, 384.2535.

### Synthesis of 5G:

3-Nitrobenzylalcohol (60.0 mg, 0.5 mmol) and purification with column chromatography on silica (eluted with DCM/MeOH gradient) produced 5G (64.0 mg, 76%). R (DCM/MeOH 9:1) 0.40; ¹H NMR (700 MHZ, CD₃OD): δ 8.21 (s, 1H), 8.13 (d, J=7.9 Hz, 1H), 7.67 (d, J=7.6 Hz, 1H), 7.54 (t, J=7.9 Hz, 1H), 5.20 (s, 2H), 2.96 (t, J=7.7 Hz, 2H), 2.38 (t, J=7.6 Hz, 2H), 1.74 (quint, J=7.6 Hz, 2H), 1.64 (quint, J=7.4 Hz, 2H), 1.36 (quint, J=7.3 Hz, 2H), 1.33-1.22 (m, 10H); ¹³C NMR (176.0 MHZ, CD₃OD): δ 173.3, 148.4, 138.3, 133.8, 129.5, 123.0, 122.7, 64.5, 40.0, 39.5, 36.8, 34.1, 29.6, 29.4, 29.3, 29.1, 29.0, 27.7, 26.9, 26.5, 26.4, 25.7, 24.8; HRMS (ESI-TOF) m/z: [M+H]⁺ calcd for C₁₈H₂₉N₂O₄, 337.2127; found, 337.2126.

### Synthesis of 5H:

4-Fluorobenzylalcohol (55.0 μL, 0.5 mmol) and purification with column chromatography on silica (eluted with DCM/MeOH gradient) produced 5H (49.0 mg, 64%). R_{f} (DCM/MeOH 9:1) 0.37; ¹H NMR (700 MHZ, CD₃OD): δ 7.31 (dd, J=8.5, 5.6 Hz, 1H), 7.02 (t, J=8.5 Hz, 1H), 5.06 (s, 2H), 2.96 (t, J=7.8 Hz, 2H), 2.31 (t, J=7.4 Hz, 2H), 1.73 (quint, J=7.5 Hz, 2H), 1.60 (quint, J=7.2 Hz, 2H), 1.34 (quint, J=7.0 Hz, 2H), 1.31-1.21 (m, 10H); ¹³C NMR (176.0 MHZ, CD₃OD): δ 173.5, 163.3, 161.9, 132.0, 130.1, 115.5, 115.3, 65.2, 40.1, 39.5, 36.8, 34.2, 29.6, 29.4, 29.3, 29.2, 29.1, 28.8, 27.7, 26.9, 26.6, 26.4, 25.7, 24.9; HRMS (ESI-TOF) m/z: [M+H]⁺ calcd for C₁₈H₂₉FNO₂, 310.2182; found, 310.2179.

### General Procedure of Derivatizing MS II (Unoptimized):

To MS II (15 mg, 0.009 mmol) in ethanol/water (1:2, 0.5 mL) were added the side chain (5D-5H) (0.018 mmol), N-methylmorpholine (NMM) (10.0 μl, 0.09 mmol), hydroxylbenzotriazole (HOBt) (8.3 mg, 0.054 mmol), and 1-ethyl-3-(3-dimethylaminopropyl) carbodiimide hydrochloride (EDC·HCl) (11.0 mg, 0.054 mmol) at room temperature. The reaction mixture was stirred for 3 days and then was directly purified with RP HPLC by using a semi-Prep C18, 250×10 mm, 5 micron column and H₂O/MeCN gradients (90%-10% H₂O over 45 minutes with a 3 mL/min flow rate). The product fraction was concentrated on a rotary evaporator at room temperature to remove MeCN, and the remaining water was then removed on a lyophilizer to provide the derivative as a white solid.

For 2D, 5D (6.3 mg, 0.018 mmol) was used to provide final product 2D (10 mg, 56%); ¹H NMR (600 MHZ, CD₃OD) (characteristicprotons): δ 9.47 (s, 1H), 6.50 (d, J=2.3 Hz, 2H), 6.42 (t, J=2.3 Hz, 1H), 5.41 (d, J=1.5 Hz, 1H), 5.30 (t, J=3.6 Hz, 1H), 5.20 (d, J=8.8 Hz, 1H), 5.05 (s, 2H), 5.02 (d, J=1.4 Hz, 1H), 4.72 (d, J=7.8 Hz, 1H), 4.56 (d, J=8.0 Hz, 1H), 4.53 (d, J=8.1 Hz, 1H), 4.50 (b, 1H), 4.44 (d, J=7.6 Hz, 1H), 4.23 (dd, J=3.0, 1.8 Hz, 1H), 3.15 (t, J=11.0 Hz, 1H), 3.13 (dd, J=9.4, 8.1 Hz, 1H), 2.88 (dd, J=14.1, 4.5 Hz, 1H), 2.38 (t, J=7.3 Hz, 2H), 2.27 (t, J=13.4 Hz, 1H), 1.16 (s, 3H), 0.99 (s, 3H), 0.92 (s, 3H), 0.86 (s, 3H), 0.77 (s, 3H); ¹³C NMR (176.0 MHZ, CD₃OD): δ 209.6, 175.5, 173.8, 169.8, 161.0, 143.5, 138.6, 121.5, 105.5, 104.7, 103.8, 103.5, 102.8, 101.9, 99.5, 99.3, 94.0, 87.4, 84.6, 84.5, 82.2, 77.3, 76.8, 76.4, 76.0, 75.4, 75.1, 74.2, 74.0, 73.6, 73.4, 73.0, 72.4, 72.3, 71.5, 70.8, 70.7, 70.5, 70.1, 70.0, 69.6, 69.2, 69.1, 68.0, 67.4, 65.7, 65.6, 60.8, 60.6, 54.8, 54.4, 41.5, 41.0, 39.7, 38.7, 38.0, 35.7, 35.3, 33.7, 32.8, 32.0, 30.5, 29.9, 29.4, 29.2, 29.1, 29.0, 28.9, 28.7, 26.5, 25.9, 24.7, 23.3, 23.1, 20.0, 17.0, 16.5, 16.4, 15.1, 9.6; HRMS (ESI-TOF) m/z: [M+H]⁺ calcd for C₉₆H₁₅₂NO₄₄, 2022.9687; found 2022.9675.

For 2E, 5E (6 mg, 0.018 mmol) was used to provide final product 2E (8 mg, 45%); ¹H NMR (600 MHZ, CD₃OD): δ 9.47 (s, 1H), 7.26 (t, J=7.9 Hz, 1H), 6.93-6.89 (m, 2H), 6.87 (dd, J=8.2, 2.7 Hz, 1H), 5.40 (d, J=1.8 Hz, 1H), 5.30 (t, J=3.7 Hz, 1H), 5.20 (d, J=8.2 Hz, 1H), 5.09 (s, 2H), 5.01 (d, J=1.6 Hz, 1H), 4.71 (d, J=7.9 Hz, 1H), 4.53 (d, J=7.9 Hz, 1H), 4.49 (b, 1H), 4.42 (d, J=7.7 Hz, 1H), 4.22 (dd, J=3.1, 1.9 Hz, 1H), 3.15 (t, J=10.8 Hz, 1H), 3.11 (dd, J=9.3, 8.0 Hz, 1H), 2.88 (dd, J=14.3, 4.1 Hz, 1H), 2.37 (t, J=7.3 Hz, 2H), 2.27 (t, J=13.6 Hz, 1H), 1.39 (s, 3H), 1.22 (d, J=6.3 Hz, 3H), 1.20 (d, J=6.5 Hz, 3H), 1.16 (s, 3H), 1.00 (s, 3H), 0.91 (s, 3H), 0.85 (s, 3H), 0.78 (s, 3H); ¹³C NMR (176.0 MHZ, CD₃OD): δ 209.6, 175.5, 173.8, 169.8, 161.4, 160.0, 143.5, 137.8, 129.2, 121.5, 119.9, 113.2, 104.7, 103.8, 103.5, 102.8, 102.7, 101.9, 99.3, 94.0, 87.4, 84.6, 84.5, 82.2, 77.3, 76.8, 76.4, 76.0, 75.5, 75.4, 75.1, 74.2, 74.0, 73.6, 73.3, 73.0, 72.4, 72.3, 71.5, 79.8, 70.7, 70.5, 70.1, 70.0, 69.6, 69.2, 69.1, 68.0, 65.7, 65.6, 60.8, 54.8, 54.3, 41.5, 39.7, 38.8, 35.7, 35.3, 33.7, 32.0, 29.9, 29.4, 29.2, 29.1, 29.0, 28.9, 28.7, 26.5, 25.9, 24.7, 23.3, 17.0, 16.5, 16.4, 15.1, 9.6; HRMS (ESI-TOF) m/z: [M+H]⁺ calcd for C₉₅H₁₄₉NO₄₃, 1992.9582; found, 1992.9662.

For 2F, 5F (7 mg, 0.018 mmol) was used to provide final product 2F (10.0 mg, 54%): ¹H NMR (600 MHZ, CD₃OD): δ 9.51 (s, 1H), 7.42-7.30 (m, 3H), 7.17 (tt, J=7.4, 2.1 Hz, 1H), 7.14 (d, J=3.7 Hz, 1H), 7.05-7.01 (m, 3H), 6.97 (dd, J=8.1, 2.3 Hz, 1H), 5.45 (d, J=1.6 Hz, 1H), 5.34 (t, J=3.5 Hz, 1H), 5.25 (d, J=8.3 Hz, 1H), 5.14 (s, 2H), 5.06 (d, J=1.5 Hz, 1H), 4.76 (d, J=7.9 Hz, 1H), 4.58 (d, J=7.9 Hz, 1H), 4.55 (b, 1H), 4.47 (d, J=7.7 Hz, 1H), 4.27 (dd, J=3.0, 1.9 Hz, 1H), 4.07-4.04 (m, 2H), 3.20 (t, J=11.0 Hz, 1H), 3.16 (dd, J=9.4, 8.0 Hz, 1H), 2.93 (dd, J=14.1, 4.4 Hz, 1H), 2.40 (t, J=7.3 Hz, 2H), 2.32 (t, J=13.6 Hz, 1H), 1.44 (s, 3H), 1.27 (d, J=6.2 Hz, 3H), 1.25 (d, J=6.4 Hz, 3H), 1.21 (s, 3H), 1.04 (s, 3H), 0.96 (s, 3H), 0.90 (s, 3H), 0.82 (s, 3H); ¹³C NMR (150.9 MHz, CD₃OD): δ 209.6, 175.5, 173.7, 169.8, 157.6, 157.1, 138.6, 129.7, 129.6, 123.2, 122.4, 121.5, 118.7, 117.9, 117.7, 104.7, 103.8, 103.5, 102.8, 101.9, 99.3, 94.0, 87.4, 84.6, 84.5, 82.2, 77.3, 76.8, 76.4, 76.0, 75.4, 75.1, 74.3, 74.2, 74.0, 73.6, 73.4, 73.0, 72.4, 72.3, 71.5, 70.8, 70.7, 70.5, 70.1, 70.0, 69.6, 69.2, 69.1, 68.0, 67.4, 65.7, 65.2, 60.8, 60.6, 54.8, 41.5, 41.0, 39.7, 38.7, 38.0, 35.7, 35.3, 33.7, 32.0, 30.5, 29.9, 29.4, 29.2, 29.1, 29.0, 28.9, 28.7, 26.5, 26.0, 24.7, 23.3, 23.1, 20.0, 17.0, 16.5, 16.4, 15.1, 9.6; HRMS (ESI-TOF) m/z: [M+H]⁺ calcd for C₁₀₀H₁₅₂NO₄₃, 2054.9738; found, 2054.9714.

For 2G, 5G (6 mg, 0.018 mmol) was used to provide final product 2G (10.0 mg, 56%): ¹H NMR (600 MHZ, CD₃OD): δ 9.51 (s, 1H), 8.29 (s, 1H), 8.24 (dd, J=8.2, 1.9 Hz, 1H), 7.82 (d, J=7.5 Hz, 1H), 7.68 (t, J=7.9 Hz, 1H), 5.44 (d, J=1.5 Hz, 1H), 5.33 (b, 1H), 5.29 (s, 2H), 5.23 (d, J=8.2 Hz, 1H), 5.05 (d, J=1.2 Hz, 1H), 4.75 (d, J=7.8 Hz, 1H), 4.57 (d, J=7.8 Hz, 1H), 4.54 (b, 1H), 4.46 (d, J=7.7 Hz, 1H), 4.26 (b, 1H), 4.07-4.02 (m, 2H), 3.19 (t, J=11.7 Hz, 1H), 3.15 (dd, J=9.4, 8.0 Hz, 1H), 2.91 (dd, J=14.0, 4.4 Hz, 1H), 2.45 (t, J=7.3 Hz, 2H), 2.30 (t, J=13.9 Hz, 1H), 1.43 (s, 3H), 1.26 (d, J=6.3 Hz, 3H), 1.24 (d, J=6.5 Hz, 3H), 1.20 (s, 3H), 1.03 (s, 3H), 0.94 (s, 3H), 0.89 (s, 3H), 0.81 (s, 3H); ¹³C NMR (176.0 MHZ, CD₃OD): δ 209.6, 175.5, 173.5, 169.6, 148.3, 143.6, 138.8, 133.9, 129.6, 124.4, 122.6, 122.3, 121.5, 104.7, 103.7, 103.5, 102.8, 101.9, 99.3, 94.0, 87.4, 84.6, 84.5, 82.1, 77.3, 76.8, 76.4, 76.0, 75.4, 75.1, 74.2, 74.0, 73.6, 73.3, 73.0, 72.4, 72.3, 71.5, 70.8, 70.7, 70.5, 70.1, 70.0, 69.6, 69.2, 69.1, 68.0, 67.4, 65.7, 64.4, 60.8, 60.6, 54.8, 41.5, 41.0, 39.7, 38.7, 38.0, 35.7, 35.3, 33.6, 32.8, 32.0, 30.5, 29.9, 29.4, 29.2, 29.1, 28.9, 28.8, 26.5, 25.9, 24.7, 23.3, 23.1, 20.0, 17.0, 16.5, 16.4, 15.1, 9.6; HRMS (ESI-TOF) m/z: [M+H]⁺ calcd for C₉₄H₁₄₇N₂O₄₄, 2007.9327; found, 2007.9337.

For 2H, 5H (5.6 mg, 0.018 mmol) was used to provide final product 2H (10.0 mg, 56%): ¹H NMR (600 MHZ, CD₃OD): δ 9.51 (s, 1H), 7.45-7.40 (m, 2H), 7.11 (tt, J=8.7, 2.1 Hz, 1H), 5.44 (d, J=1.6 Hz, 1H), 5.34 (t, J=3.4 Hz, 1H), 5.24 (d, J=8.3 Hz, 1H), 5.13 (s, 2H), 5.05 (d, J=1.6 Hz, 1H), 4.75 (d, J=8.0 Hz, 1H), 4.57 (d, J=7.8 Hz, 1H), 4.54 (b, 1H), 4.46 (d, J=7.6 Hz, 1H), 4.26 (dd, J=3.0, 2.1 Hz, 1H), 3.19 (t, J=11.0 Hz, 1H), 3.15 (dd, J=9.2, 8.3 Hz, 1H), 2.92 (dd, J=14.5, 4.4 Hz, 1H), 2.39 (t, J=7.5 Hz, 2H), 2.31 (t, J=13.6 Hz, 1H), 1.44 (s, 3H), 1.26 (d, J=6.3 Hz, 3H), 1.25 (d, J=6.4 Hz, 3H), 1.21 (s, 3H), 1.04 (s, 3H), 0.95 (s, 3H), 0.89 (s, 3H), 0.82 (s, 3H); ¹³C NMR (176.0 MHZ, CD₃OD): δ 209.6, 175.5, 173.7, 169.8, 163.3, 161.9, 143.6, 132.5, 130.2, 130.1, 114.9, 114.8, 104.7, 103.7, 103.5, 102.8, 101.9, 99.3, 94.0, 87.4, 84.6, 84.5, 82.2, 77.3, 76.8, 76.4, 76.0, 75.4, 75.1, 74.2, 74.0, 73.6, 73.3, 73.0, 72.4, 72.3, 71.5, 70.8, 70.7, 70.5, 70.1, 70.0, 69.6, 69.2, 69.1, 68.0, 67.4, 65.7, 65.0, 60.8, 60.6, 54.8, 48.5, 41.5, 41.0, 39.7, 38.7, 38.0, 35.7, 35.3, 33.7, 32.8, 32.0, 30.5, 29.9, 29.4, 29.2, 29.1, 29.0, 28.9, 28.7, 26.5, 25.9, 24.7, 23.3, 23.1, 20.0, 17.0, 16.5, 16.4, 15.1, 9.6; HRMS (ESI-TOF) m/z: [M+H]⁺ calcd for C₉₄H₁₄₇FNO₄₂, 1980.9382; found, 1980.9463.

### Antigens:

The chicken egg albumin for in vivo use (Vac-pova) was purchased from InvivoGen.

### Mice and Immunization:

BALB/c mice used in this study were from Frederick Cancer Research (Fredrick, MD). To assess the adjuvant activity of the MS saponin-based immune adjuvants, groups of female mice (8-10 weeks of age; 6 mice per group) were immunized by the subcutaneous (s.c.) route with OVA (20 μg) alone, or with antigen plus proper adjuvant such as QS-21 (20 μg) or a MS adjuvant (50-100 μg) on days 0, 14 and 28. Prior to each immunization and at two weeks post last immunization, mice were weighed and blood samples were collected from the lateral tail vein by using heparinized capillary pipettes. The serum was obtained after centrifugation and stored at −20° C. until assayed.

### Evaluation of Antibody Responses:

The levels of specific serum IgG and IgG subclasses against OVA in each group were determined by an enzyme-linked immunosorbent assay (ELISA). Maxisorpmicrotiter plates (NUNC International, Roskilde, DK) were coated with rHagB (1 μg/ml), OVA (0.1 μg/ml), or with optimal amounts of goat anti-mouse IgG, IgG1 or IgG2a in borate buffer saline (BBS; 100 mM NaCl, 50 mM boric acid, 1.2 mM Na₂B₄O₇, PH 8.2) at 4° C. overnight. Plates were blocked with 1% bovine serum albumin (BSA) and 0.02% sodium azide in BBS for 2 h at room temperature. Serial two-fold dilutions of serum samples were added in duplicate to the plates. To generate standard curves, serial dilutions of a mouse immunoglobulin reference serum (MP Biomedicals, Solon, OH) were added to two rows of wells in each plate that had been coated with the appropriate anti-mouse IgG or IgG subclass reagent. After incubation (overnight at 4° C.) and washing of the plates, horseradish peroxidase-conjugated goat anti-mouse IgG or IgG subclass antibody was added to appropriate wells. After 4 h of incubation at room temperature, plates were washed and developed by o-phenylenediamine substrate with hydrogen peroxide. Color development was recorded at 490 nm. The concentrations of antibodies were determined by interpolation on standard curves generated by using the mouse immunoglobulin reference serum and constructed by a computer program based on four-parameter logistic algorithms (Softmax/Molecular Devices Corp., Menlo Park, CA).

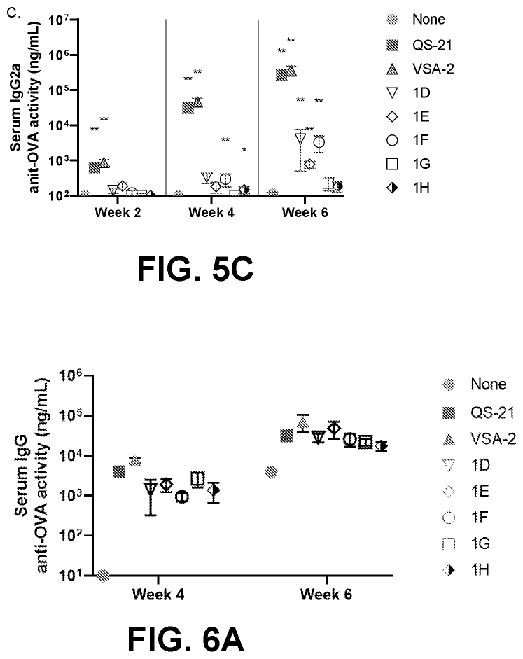

FIGS. 5A-5C illustrate serum IgG, IgG1, and IgG2a anti-OVA responses in mice immunized by the subcutaneous route with ovalbumin (OVA) alone, or with QS-21 or VSA-2, or with one of the saponin adjuvants 1D-1H. Statistical significance was evaluated by t tests (with unpaired, nonparametric and Mann-Whiteny test). *P<0.05, **P<0.01.

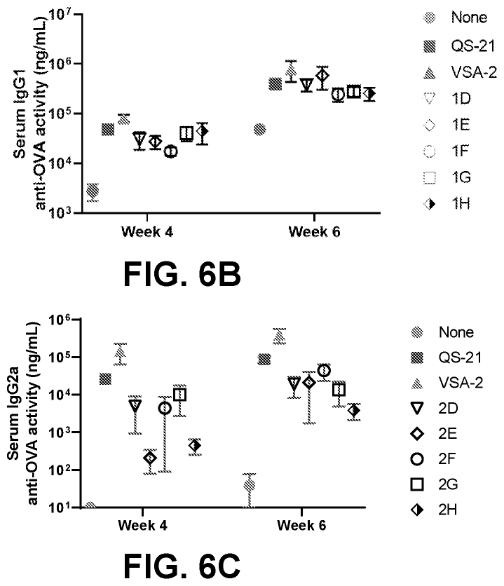

FIGS. 6A-6C illustrate serum IgG, IgG1, and IgG2a anti-OVA responses in mice immunized by the subcutaneous route with ovalbumin (OVA) alone, or with QS-21 or VSA-2, or with one of the saponin adjuvants 2D-2H.

### Statistical Analysis:

Statistical significance in antibody responses was evaluated by t tests (with unpaired, nonparametric and Mann-Whiteny test) using GraphPad Prism 8. Differences were considered significant at a P value <0.05.

### CONCLUSION

A number of MS saponin-derived immunostimulants have been prepared and evaluated. These MS derivatives were prepared by incorporating a terminal-functionalized side chain into the C3 glucuronic acid unit of the natural saponin MS II through amide formation reaction. These unnatural semisynthetic saponins showed potent immunostimulant activity profiles comparable with the FDA-approved saponin immunostimulant QS-21.

### REFERENCES FOR EXAMPLE 1

1. Brunner R, Jensen-Jarolim E, Pali-Scholl I. The ABC of clinical and experimental adjuvants—a brief overview. Immunol Lett. 2010; 128(1): 29-35. doi: 10.1016/j.imlet.2009.10.005. PubMed PMID: 19895847; PMCID: 2999744.
2. Kensil C R, Mo A X, Truneh A. Current vaccine adjuvants: An overview of a diverse class. Frontiers in Bioscience. 2004:9:2972-88.
3. Leroux-Roels G. Unmet needs in modern vaccinology: adjuvants to improve the immune response. Vaccine. 2010; 28 Suppl 3: C25-36. doi: 10.1016/j.vaccine.2010.07.021. PubMed PMID: 20713254.
4. Sharp F A, Lavelle E C. Discovery of vaccine adjuvants. In: Gad S C, editor. Development of Therapeutic Agents Handbook. 1st ed. Hoboken, NJ: John Wiley & Sons, Inc: 2012. p. 533-46.
5. Wang W, Singh M. Selection of adjuvants for enhanced vaccine potency. World J Vaccines. 2011; 01(02):33-78. doi: 10.4236/wjv.2011.12007.

6 Weeratna R D, McCluskie M J. Recent advances in vaccine adjuvants. In: Miller A A, Miller P F, editors. Emerging Trends in Antibacterial Discovery: Answering the Call to Arms. Great Britain: Caister Academic Press; 2011. p. 303-22.
7. Coffman R L, Sher A, Seder R A. Vaccine adjuvants: putting innate immunity to work. Immunity. 2010; 33(4): 492-503. doi: 10.1016/j.immuni.2010.10.002. PubMed PMID: 21029960; PMCID: 3420356.
8. Banday A H, Jeelani S, Hruby V J. Cancer vaccine adjuvants—recent clinical progress and future perspectives. Immunopharmacol Immunotoxicol. 2015; 37(1):1-11. Epub 2014 Oct. 17. doi: 10.3109/08923973.2014.971963. PubMed PMID: 25318595.
9. Bastola R, Noh G, Keum T, Bashyal S, Seo J E, Choi J, Oh Y, Cho Y, Lee S. Vaccine adjuvants: smart components to boost the immune system. Archives of pharmacal research. 2017; 40(11): 1238-48. Epub 2017 Oct. 14. doi: 10.1007/s12272-017-0969-z. PubMed PMID: 29027637.
10. Di Pasquale A, Preiss S, Tavares Da Silva F, Garcon N. Vaccine Adjuvants: from 1920 to 2015 and Beyond. Vaccines (Basel). 2015; 3(2):320-43. Epub 2015 Sep. 8. doi: 10.3390/vaccines3020320. PubMed PMID: 26343190; PMCID: PMC4494348.
11. Temizoz B, Kuroda E, Ishii K J. Vaccine adjuvants as potential cancer immunotherapeutics. Int Immunol. 2016; 28(7):329-38. Epub 2016 Mar. 24. doi: 10.1093/intimm/dxw015. PubMed PMID: 27006304; PMCID: PMC4922024.
12. Bonam S R, Partidos C D, Halmuthur S K M, Muller S. An overview of novel adjuvants designed for improving vaccine efficacy. Trends in Pharmacological Sciences. 2017; 38(9):771-93.
13. O'Hagan D T, Friedland L R, Hanon E, Didierlaurent A M. Towards an evidence based approach for the development of adjuvanted vaccines. Current Opinion in Immunology. 2017; 47:93-102.
14. Garçon N, Leroux-Roels G, Cheng W-F. Vaccine Adjuvants. In: Garçon N, Stern P L, Cunningham A L, editors. Understanding Modern Vaccines Perspectives in Vaccinology Amsterdam: Elsevier; 2011. p. 89-113.
15. Giudice G D, Rappuoli R, Didierlaurent A M. Correlates of adjuvanticity: A review on adjuvants in licensed vaccines. Semin Immunol. 2018; 2018:14-21.
16. Shi S, Zhu H, Xia X, Liang Z, Ma X, Sun B. Vaccine adjuvants: Understanding the structure and mechanism of adjuvanticity. Vaccine. 2019; 37:3167-78.
17. Rivera-Hernandez T, Pandey M, Henningham A, Cole J, Choudhury B, Cork A J, Gillen C M, Ghaffar K A, West N P, Silvestri G, Good M F, Moyle P M, Toth I, Nizet V, Batzloff M R, Walker M J. Differing Efficacies of Lead Group A Streptococcal Vaccine Candidates and Full-Length M Protein in Cutaneous and Invasive Disease Models. mBio. 2016; 7(3):e00618-16. doi: 10.1128/mBio.00618-16.
18. Rivera-Hernandez T, Rhyme M S, Cork A J, Jones S, Segui-Perez C, Brunner L, Richter J, Petrovsky N, Lawrenz M, Goldblatt D, Collin N, Walker M J. Vaccine-Induced Th1-Type Response Protects against Invasive Group A Streptococcus Infection in the Absence of Opsonizing Antibodies. mBio. 2020; 11(2). Epub 2020 Mar. 12. doi: 10.1128/mBio.00122-20. PubMed PMID: 32156809: PMCID: PMC7064752.
19. Didierlaurent A M, Laupeze B, Di Pasquale A, Hergli N, Collignon C, Garcon N. Adjuvant system AS01: helping to overcome the challenges of modern vaccines. Expert Rev Vaccines. 2017; 16(1):55-63. Epub 2016 Jul. 28. doi: 10.1080/14760584.2016.1213632. PubMed PMID: 27448771.
20. James S F, Chahine E B, Sucher A J, Hanna C. Shingrix: the new adjuvanted recombinant herpes zoster vaccine. Annals of Pharmacotherapy. 2018; 52(7):673-80.
21. Cunningham A L, Lal H, Kovac M, Chlibek R, Hwang S-J, Diez-Domingo J, Godeaux O, Levin M J, McElhaney J E, Puig-Barberà J, VandenAbeele C, Vesikari T, Watanabe D, Zahaf T, Ahonen A, Athan E, Barba-Gomez J F, Campora L, deLooze F, Downey H J, Ghesquiere W, Gorfinkel I, Korhonen T, Leung E, McNeil S A, Oostvogels L, Rombo L, Smetana J, Weckx L, Yeo W, Heineman T C. Efficacy of the Herpes Zoster Subunit Vaccine in Adults 70 Years of Age or Older. The New England Journal of Medicine. 2016; 375(11):1019-32.
22. Lacaille-Dubois M A. Updated insights into the mechanism of action and clinical profile of the immunoadjuvant QS-21: A review. Phytomedicine. 2019; 60:152905. Epub 2019 Jun. 12. doi: 10.1016/j.phymed.2019.152905. PubMed PMID: 31182297; PMCID: PMC7127804.
23. Martin R S, Briones R. Industrial uses and sustainable supply of Quillaja saponaria (Rosaceae) Saponins. Economic Botany. 1999; 53(3):302-11.
24. Ragupathi G, Gardner J R, Livingston P O, Gin D Y. Natural and synthetic saponin adjuvant QS-21 for vaccines against cancer Expert Rev Vaccines. 2011; 10:463-70.
25. Wang P, Dai Q, Thogaripally P, Zhang P, Michalek S M. Synthesis of QS-21-based immunoadjuvants. J Org Chem. 2013; 78:11525-34.
26. Wang P, Devalankar D A, Dai Q, Zhang P, Michalek S M. Synthesis and Evaluation of QS-21-Based Immunoadjuvants with a Terminal-Functionalized Side Chain Incorporated in the West Wing Trisaccharide. J Org Chem. 2016; 81:9560-6.
27. Wang P, Ding X, Kim H, Škalamera Ð, Michalek S M, Zhang P. Vaccine adjuvants derivatized from Momordica saponins I and II. J Med Chem. 2019; 62:9976-82.
28. Wang P, Škalamera Ð, Sui X, Zhang P, Michalek S M. Synthesis and evaluation of a QS-17/18-based vaccine adjuvant. J Med Chem. 2019; 62:1669-76. doi: 10.1021/acs.jmedchem.8b01997.
29. Wang P, Škalamera Ð, Sui X, Zhang P, Michalek S M. Synthesis and evaluation of QS-7-based vaccine adjuvants. ACS Infectious Diseases. 2019; 5:974-81. doi: 10.1021/acsinfecdis.9b00039.
30. Wang P. Natural and Synthetic Saponins as Vaccine Adjuvants. Vaccines (Basel). 2021; 9(3):222. doi: https://doi.org/10.3390/vaccines9030222.
31. Wang P, Ding X, Kim H, Michalek S M, Zhang P. Structural Effect on Adjuvanticity of Saponins. J Med Chem. 2020; 63:3290-7.

1-3. (canceled)

4. A pharmaceutical composition comprising a therapeutically effective amount of a modified saponin having the formula, to treat a condition: 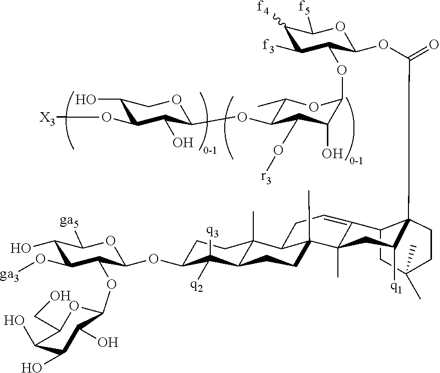
wherein:
q₁ is H or OH;
q₂ and q₃ are each independently selected from CHO, CH₃, CH₂OH, H, COOH, a component of an acetal group, or an imine group;
f₃ and f₄ are each independently OH or an acetyl, or C3 and C4 of a fuocsyl unit wherein f₃, and f₄ can form a cyclic ketal ring or cyclic carbonate ester;
f₅ is selected from H, methyl group, —CH₂OH group, R₄—NR₅—C(O)—, R₄—O—C(O)—, or R₄—O—CH₂—;
wherein R₄ and R₅ are each independently a linear chain having the structure R₆[(CX₂)₀₋₂₀O₀₋₁(CY₂)₀₋₂₀]₀₋₂₀;
wherein X and Y are each independently H or a halogen atom;
wherein Re are COOR₇, C(O)NR₇R₈, NR₇R₈, or OR₇;
wherein R₇ are Ar[(CZ₂)₀₋₂₀O₀₋₁(CL₂)₀₋₂₀]₀₋₂₀, wherein R₈ is H or an alkyl;
wherein Z and L are each independently H or a halogen atom;
wherein Ar is a substituted or unsubstituted aromatic system;
ga₅ is selected from R₁₄—NR₁₅—C(O)—, R₁₄—O—C(O)—, or R₁₄—O—CH₂—;
wherein R₁₄ and R₁₅ are each independently a linear chain having the structure R₁₆[(CX1₂)₀₋₂₀O₀₋₁(CY1₂)₀₋₂₀]₀₋₂₀;
wherein X1 and Y1 are each independently H or a halogen atom;
wherein R₁₆ is COOR₁₇, C(O)NR₁₇R₁₈, NR₁₇R₁₈, or OR₁₇;
wherein R₁₇ is Ar2[(CZ1₂)₀₋₂₀O₀₋₁(CL1₂)₀₋₂₀]₀₋₂₀, wherein R₁₈ is H or an alkyl;
wherein Z1 and L1 are each independently H or a halogen atom;
wherein Ar2 is a substituted or unsubstituted aromatic group;
r3 is H, a monosaccharide, disaccharide, or a trisaccharide;
x3 is H, a monosaccharide (except xylose) or a disaccharide; and
ga3 is H, a monosaccharide or a disaccharide.

5. A pharmaceutical composition comprising a therapeutically effective amount of a modified saponin having the formula I, to treat a condition: 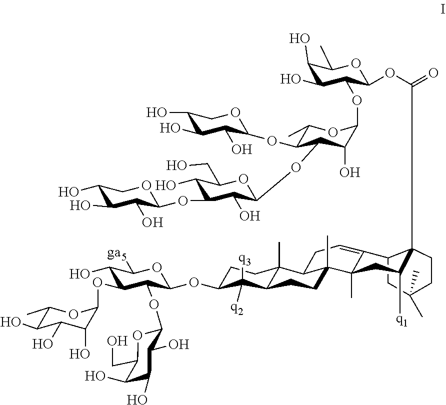
wherein:
q₁ is H or OH;
q₂ and q₃ are each independently selected from CHO, CH₃, CH₂OH, H, COOH, a component of an acetal group, or an imine group;
ga₅ is selected from R₁₄—NR₁₅—C(O)—, R₁₄—O—C(O)—, or R₁₄—O—CH₂—;
wherein R₁₄ and R₁₅ are each independently a linear chain having the structure R₁₆[(CX1₂)₀₋₂₀O₀₋₁(CY1₂)₀₋₂₀]₀₋₂₀;
wherein X1 and Y1 are each independently H or a halogen atom;
wherein R₁₆ is COOR₁₇, C(O)NR₁₇R₁₈, NR₁₇R₁₈, or OR₁₇;
wherein R₁₇ is Ar2[(CZ1₂)₀₋₂₀O₀₋₁(CL1₂)₀₋₂₀]₀₋₂₀, wherein R₁₈ is H or an alkyl;
wherein Z1 and L1 are each independently H or a halogen atom; and
wherein Ar2 is a substituted or unsubstituted aromatic group.

6. A pharmaceutical composition comprising a therapeutically effective amount of a modified saponin having one of formulas 2D-H, to treat a condition:

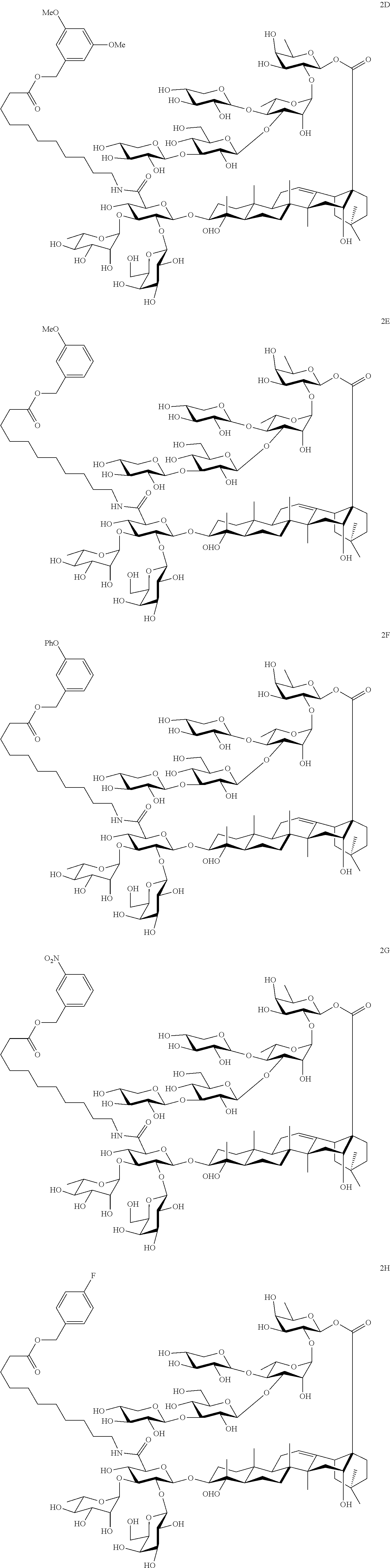

7. The pharmaceutical composition of claim 6, wherein the composition further comprises at least one immunogen.

8. The pharmaceutical composition of claim 6, wherein the composition further comprises a pharmaceutically acceptable carrier.

9. The pharmaceutical composition of claim 6, the composition is formulated for administering to an animal or human subject.

10. The pharmaceutical composition of claim 6, wherein the composition further comprises at least one cancer therapeutic agent, wherein the at least one chemotherapeutic agent and the saponin derivative are admixed in a pharmaceutically acceptable formulation or covalently linked to each other, and a pharmaceutically acceptable carrier.

11-14. (canceled)

15. A method of treating a condition comprising: administering to a subject in need thereof, a pharmaceutical composition, wherein the pharmaceutical composition includes a therapeutically effective amount of the pharmaceutical composition of claim 6.

16. The pharmaceutical composition of claim 4, wherein the composition further comprises at least one immunogen.

17. The pharmaceutical composition of claim 4, wherein the composition further comprises a pharmaceutically acceptable carrier.

18. The pharmaceutical composition of claim 4, the composition is formulated for administering to an animal or human subject.

19. The pharmaceutical composition of claim 4, wherein the composition further comprises at least one cancer therapeutic agent, wherein the at least one chemotherapeutic agent and the saponin derivative are admixed in a pharmaceutically acceptable formulation or covalently linked to each other, and a pharmaceutically acceptable carrier.

20. The pharmaceutical composition of claim 5, wherein the composition further comprises at least one immunogen.

21. The pharmaceutical composition of claim 5, wherein the composition further comprises a pharmaceutically acceptable carrier.

22. The pharmaceutical composition of claim 5, the composition is formulated for administering to an animal or human subject.

23. The pharmaceutical composition of claim 5, wherein the composition further comprises at least one cancer therapeutic agent, wherein the at least one chemotherapeutic agent and the saponin derivative are admixed in a pharmaceutically acceptable formulation or covalently linked to each other, and a pharmaceutically acceptable carrier.
